(19) United States
(12) Patent Application Publication
(10) Pub. No.: US 2013/0245253 A1
(43) Pub. Date: Sep. 19, 2013

(54) Conjugated Neuroactive Steroid Compositions And Methods Of Use

(71) Applicants: Christine Marx, Durham, NC (US); Robert Mook, Durham, NC (US)

(72) Inventors: Christine Marx, Durham, NC (US); Robert Mook, Durham, NC (US)

(73) Assignees: DEPARTMENT OF VETERANS AFFAIRS, Baltimore, MD (US); DUKE UNIVERSITY, Durham, NC (US)

(21) Appl. No.: 13/637,617

(22) PCT Filed: Mar. 28, 2011
(86) PCT No.: PCT/US11/30201

§ 371 Date: Nov. 27, 2012

Related U.S. Application Data
(60) Provisional application No. 61/317,989, filed on Mar. 26, 2010.

(51) Int. Cl. C07J 7/00; C07J 43/00

(52) U.S. Cl. CPC C07J 7/001; C07J 43/003
USPC 540/113; 552/509; 552/606

(57) ABSTRACT

The present disclosure provides modified neuroactive steroids. The modified neuroactive steroids may comprise, consist of, or consist essentially of a therapeutic agent and/or a modifying moiety. The modified neuroactive steroid can have modified characteristics as compared to native neuroactive steroids that do not include a modifying moiety and/or therapeutic agent. The modified neuroactive steroid may be, for example, modified pregnenolone, pregnenolone metabolites, allopregnanolone, and/or allopregnanolone metabolites. The modified neuroactive steroids can be used to treat, prevent and/or ameliorating a phenotypic state of interest in a subject.

### STATEMENT REGARDING FEDERALLY SPONSORED RESEARCH & DEVELOPMENT

The present disclosure was made with U.S. Government support under the following grants: VA Research Career Development Transition Award (CDTA), VA Advanced Research Career Development Award (ARCD) VA, Mid-Atlantic Mental Illness Research Education and Clinical Center (MIRECC).

### TECHNICAL FIELD

The present disclosure relates to conjugated neuroactive steroid compositions and methods of treating, ameliorating and/or preventing neurological and/or psychiatric disorders and nervous system lesions and/or medical conditions in a subject using said compositions.

### BACKGROUND OF THE DISCLOSURE

Healthcare, including most psychiatric, neurological, and medical care, is largely organized around prescribing medication to manage illness. Patient compliance (also referred to herein as adherence) with medications, however, remains a serious problem. Poor adherence to medications accounts for an estimated 33% to 69% of medication-related hospital admissions, costing about $100 billion (Osterberg L and Blaschke L. N Engl J Med. 2005; 353[5]:487-497). Harder to measure, but no less real, are the premature deaths and reduced quality of life for patients; increased patient and physician anger, frustration, and hopelessness; and potentially skewed findings for clinical research.

Noncompliance with medications can stem from many factors, including complex and often long-term drug regimens that alter existing behavioral patterns and/or have unwanted side effects (sometimes due to required therapeutic dosage amounts), as well as medication that is prescribed as part of a primary prevention strategy, such as the use of statins for lowering cholesterol levels to reduce cardiovascular risk (Mitka, M. (2010) JAMA 9:825). In addition, children are less likely than adults to follow a treatment plan because of their dependence on an adult caregiver (Compliance. The Merck Manual of Diagnosis and Therapy, section 19, chapter 258, Drug Treatment in Newborns, Infants, and Children. Available at www.merck.com).

Patient compliance is particularly a concern for those afflicted with psychiatric and/or neurological disorders and/or medical conditions, such as schizophrenia, depression, traumatic brain injury (TBI), lipid disorders, post-traumatic stress disorder (PTSD), bipolar disorder, substance use disorders (including nicotine dependence), Alzheimer's disease (and other disorders in which cognition is impaired), and pain disorders, among others. Many patients experience outcomes that are frequently compromised by therapeutics with suboptimal pharmacokinetic properties and/or challenging dosing requirements and/or negative side effects and/or are characterized by the development of dependence and/or tolerance. Further, effective pharmacological interventions for many psychiatric and neurological disorders are currently lacking or therapeutically suboptimal, with large numbers of patients remaining persistently symptomatic and refractory to pharmacological intervention with currently available agents. Since many currently available agents produce only partial symptom reduction, novel conjugation strategies with other molecules could increase therapeutic efficacy and optimize clinical response. New pharmacological interventions that are well-tolerated, easily administered, effective, and likely to produce enhanced compliance (resulting in improved long-term outcome and quality of life) are thus urgently needed for many psychiatric, neurologic, and medical disorders.

Therefore, there is a critical need for improved pharmaceutical compositions that will increase patient compliance and demonstrate enhanced therapeutic efficacy.

### SUMMARY OF THE DISCLOSURE

The present disclosure broadly comprises variant and modified forms of several naturally occurring neuroactive steroids, analogs, chemical conjugates, pharmaceutically acceptable salts thereof, derivatives thereof, or combinations thereof that possess one or more advantages over their naturally occurring counterparts. By way of example, some of these advantages include an increased resistance to metabolic breakdown (e.g., proteolytic degradation), an improved time of persistence in the bloodstream (e.g., sustained release), additional pharmacologic activity (e.g., increased biologic levels of the neuroactive steroid); and/or the ability to target specific cells/organs of a subject.

Modified neuroactive steroids according to some embodiments of the present disclosure comprise, consist of, or consist essentially of a modified neuroactive steroid having the general formula R-Lₙ-NS, wherein R is H, a therapeutic agent, or a modifying moiety, L is a linking group, n is an integer of 0 to 50, wherein the integer represents the number of linking groups, and NS is a neuroactive steroid. By virtue of the modifying moiety or therapeutic agent attached to the modified neuroactive steroid, the modified neuroactive steroid can have modified characteristics as compared to native neuroactive steroids that do not include a modifying moiety as described herein.

In one embodiment, n is 0, and the modified neuroactive steroid has the following structure (Formula I):

wherein:

R₁ is —OH, a sulfate, a phosphate, or a modifying moiety;

R₂ is ═O, —OH, or a modifying moiety, and
denotes an optional C═C bond, with the proviso that there is not a C═C bond between both C17-C20 and C20-C21;

with the proviso that at least one of R₁ and R₂ is a modifying moiety.

In another embodiment, n is 0, and the modified neuroactive steroid has the following structure (Formula II):

wherein:

R₁ is —OH, ═O, a sulfate, a phosphate, or a modifying moiety;

R₂ is ═O, —OH, or a modifying moiety, and
denotes an optional C═C bond, with the proviso that there is not a C═C bond between both C17-C20 and C20-C21;

with the proviso that at least one of R₁ and R₂ is a modifying moiety.

In yet another embodiment, n is 0, and the modified neuroactive steroid has the following structure (Formula III):

wherein:

R₁ is —OH, a sulfate, a phosphate, or a modifying moiety;

R₂ is ═O or a modifying moiety; and
denotes an optional C═C bond;

with the proviso that at least one of R₁ and R₂ is a modifying moiety.

In still another embodiment, n is 0 and the modified neuroactive steroid has the following structure (Formula IV):

wherein:

R₁ is ═O, —OH, a sulfate, a phosphate, or a modifying moiety,

R₂ is ═O, —OH, or a modifying moiety,

R₃ is —H, —OH, or a modifying moiety,

R₄, when present, is —H, —OH, or a modifying moiety, and

R₅ is —OH or a modifying moiety, and
denotes an optional C═C bond;

with the proviso that at least one of R₁, R₂, R₃, R₄, and R₅ is a modifying moiety.

In other embodiments, the present disclosure is directed to a modified neuroactive steroid having the general formula R-Lₙ-NS, wherein R is H, a neuroactive steroid, a therapeutic agent, or a modifying moiety, L is a linker, n is an integer that is equal to, or greater than, 1, and NS is a neuroactive steroid.

In one particular embodiment, the present disclosure is directed to a modified neuroactive steroid having a structure selected from the group consisting of Formulas V-VII:

wherein R is selected from a neuroactive steroid, H, a therapeutic agent, a modifying moiety, and combinations thereof; L is a linker; n is an integer equal to, or greater than, 1; R₂ is ═O or —OH; R₃ is —H or —OH; R₄ is —H or —OH, and is an optional C═C bond.

In one particular embodiment, the neuroactive steroid is pregnenolone. In such embodiments, the modified neuroactive steroid may comprises, consists of, or consists essentially of the following formula:

In some cases, the modified neuroactive steroid is characterized at least in part by its increased resistance to metabolism, such as conjugation, proteolysis or hydrolysis, the ability to tune the rate of hydrolysis, and the ability to provide sustained amounts of the modified neuroactive steroid over time by controlling the rate of drug/steroid release, relative to a corresponding unmodified form of the native neurosteroid compound. In other cases, the modified neuroactive steroid is characterized at least in part by its ability to target a desired tissue, organ and/or cell type. Still in other cases, the modified neuroactive steroid is characterized by enhanced or additional pharmacological activity as compared to a corresponding unmodified form of the endogenous neurosteroid compound. These modified neuroactive steroids may even be further characterized by a retained therapeutically significant percentage of biological activity, such as NMDA and/or GABA_{A} receptor inhibiting or enhancing activity, relative to the endogenous, unmodified neuroactive steroids.

The present disclosure also provides several methods for the preparation of the modified neuroactive steroids. These modifying moieties, can take a number of different forms, such as simple ester derivatives of carboxylic acids, known compounds, such as drug or prodrug compositions, pharmacologic agents, and the like that impart a desired effect, such as sustained release, tissue targeting, or additional pharmacologic activity.

In other aspects, the present disclosure provides method of treating, preventing and/or ameliorating a phenotypic state of interest in a subject comprising, consisting of, or consisting essentially of administering a therapeutically effective amount of a modified neuroactive steroid according to the present disclosure such that the phenotypic state of interest is treated, prevented or ameliorated in the subject.

Other advantages and novel features of the present disclosure will become apparent from the following detailed description of various non-limiting embodiments of the disclosure when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### DETAILED DESCRIPTION OF THE DISCLOSURE

The present disclosure is not limited in its application to the details of construction and the arrangement of components set forth in the following description or illustrated in the drawings. The invention described in the present disclosure is capable of other embodiments and of being practiced or of being carried out in various ways. Also, the phraseology and terminology used herein is for the purpose of description and should not be regarded as limiting. The use of “including,” “comprising,” “having,” “containing,” “involving,” and variations thereof herein, is meant to encompass the items listed thereafter and equivalents thereof as well as additional items. For the purposes of promoting an understanding of the principles of the present disclosure, reference will now be made to preferred embodiments and specific language will be used to describe the same.

Articles “a” and “an” are used herein to refer to one or to more than one (i.e. at least one) of the grammatical object of the article. By way of example, “an element” means at least one element and can include more than one element.

### DEFINITIONS

Unless otherwise defined, all technical and scientific terms used herein have the same meaning as commonly understood by one of ordinary skill in the art to which the invention belongs. The terminology used in the description of the invention herein is for the purpose of describing particular embodiments only and is not intended to be limiting of the invention. Headers are used for the convenience of the reader and are also not intended to be limiting of the invention. All publications, patent applications, patents, and other references mentioned herein are incorporated herein by reference in their entirety, as are the package inserts of any branded drugs referred to herein by their brand names.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. Preferably, the subject is a human patient.

In accordance with the present disclosure, the subject being treated comprises a phenotypic state of interest. Examples of phenotypic states include, for example, phenotypes resulting from an altered environment, drug treatment, genetic manipulations or mutations, injury, change in diet, aging, or any other characteristic(s) of a single organism or a class or subclass of organisms. In certain embodiments, a phenotypic state of interest is a clinically diagnosed disease state or disorder. Such disease states and/or disorders include, for example, cancer, cardiovascular disease, inflammatory disease, autoimmune disease, neurological/psychiatric disease, pulmonary disorders, gastrointestinal disorders, metabolic disorders (such as diabetes, among others), blood disorders (such as coagulopathies, among others), renal disease, endocrine disorders, dermatological conditions, infectious disease, pregnancy-related disorders (e.g., post-partum depression), and the like, and combinations thereof. In one embodiment, the phenotypic state of interest is a neurological/psychiatric disease. It certain embodiments, the subject may be affected by one, two, three or more phenotypic states at one time, and may be treated for one, two, three or more of such conditions accordingly.

As used herein, the term “neurological or psychiatric disorder” and “disorder(s) of the nervous system” are used interchangeably herein and refers broadly to any disorder of emotional, mental, physical, personality, and/or mental function that is of neurological, psychiatric, psychological, medical, or mixed origin that negatively impacts the emotional mental, physical, social, occupational, and/or cognitive functioning of a subject. Some representative neurological and psychiatric disorders include those listed in the Diagnostic and Statistical Manual of Mental Disorders (DSM; including DSM-IV, DSM-IV-TR, and/or the upcoming DSM-V). Examples of such disorders include, but are not limited to, substance use disorders (e.g., use, abuse, and/or dependence on cocaine, opioid, cannabis, amphetamine, alcohol, caffeine, tobacco/nicotine, hallucinogens and the like, and withdrawal or related conditions); anxiety disorders (e.g., post-traumatic stress disorder, obsessive compulsive disorder, panic disorder, agoraphobia, social phobia, acute stress disorder, generalized anxiety disorder, substance-induced anxiety disorder and the like); mood disorders (e.g., both depressive and manic disorders including but not limited to major depressive disorder, major depressive disorder with psychotic features, major depressive disorder with post-partum onset, dysthymic disorder, bipolar I disorder, bipolar II disorder, cyclothymic disorder, substance-induced mood disorder and the like); psychotic disorders (e.g., schizophrenia, schizoaffective disorder, delusional disorder, brief psychotic disorder, shared psychotic disorder, psychotic disorder due to a medical condition, substance-induced psychotic disorder, psychotic disorder not otherwise specified, and the like); cognitive disorders (e.g., mild cognitive impairment, Alzheimer's disease, vascular dementia, dementia due to other medical conditions, dementia due to multiple etiologies, substance-induced persisting amnestic disorder, amnestic disorder not otherwise specified, delirium and the like); and other disorders, such as brain injury-related disorders, such as traumatic brain injury (TBI) (including concussion); hypoxia due to stroke/hemorrhage/aneurism, multiple sclerosis, amyotrophic lateral sclerosis (ALS), attention deficit hyperactivity disorder (ADHD) (all types), pain disorders, Niemann-Pick type C disease, conditions related to multiple sclerosis (MS) and other neurological disorders such as Parkinson's disease (e.g., cognitive symptoms, depression, among others), and the like.

Cardiovascular disease may be treated in other applications of the present disclosure. Examples of cardiovascular disease include, but are not limited to, congestive heart failure, high blood pressure (e.g., hypertension), arrhythmias, atherosclerosis, lipid disorders (e.g., high cholesterol and/or triglycerides, and the like), Wolff-Parkinson-White Syndrome, long QT syndrome, angina pectoris, tachycardia, bradycardia, atrial fibrillation, ventricular fibrillation, congestive heart failure, myocardial ischemia, myocardial infarction, cardiac tamponade, myocarditis, pericarditis, arrhythmogenic right ventricular dysplasia, hypertrophic cardiomyopathy, Williams syndrome, heart valve diseases, endocarditis, bacterial, pulmonary atresia, aortic valve stenosis, Raynaud's disease, Raynaud's disease, cholesterol embolism, Wallenberg syndrome, Hippel-Lindau disease, stroke and telangiectasis.

Inflammatory disease and autoimmune disease may be treated in other applications of the present disclosure. Examples of inflammatory disease and autoimmune disease include, but are not limited to, rheumatoid arthritis, non-specific arthritis, inflammatory disease of the larynx, inflammatory bowel disorder, psoriasis, hypothyroidism (e.g., Hashimoto thyroidism), colitis, Type 1 diabetes, pelvic inflammatory disease, inflammatory disease of the central nervous system, temporal arteritis, polymyalgia rheumatica, ankylosing spondylitis, polyarteritis nodosa, Reiter's syndrome, scleroderma, systemis lupus, multiple sclerosis and erythematosus.

Cancer phenotypes that may also be treated in accordance to the present disclosure. Examples of such cancer phenotypes include, but are not limited to: breast cancer, skin cancer, bone cancer, prostate cancer, liver cancer, lung cancer, brain cancer, cancer of the larynx, gallbladder, pancreas, rectum, parathyroid, thyroid, adrenal, neural tissue, head and neck, colon, stomach, bronchi, kidneys, basal cell carcinoma, squamous cell carcinoma of both ulcerating and papillary type, metastatic skin carcinoma, osteo sarcoma, Ewing's sarcoma, veticulum cell sarcoma, myeloma, giant cell tumor, small-cell lung tumor, non-small cell lung carcinoma gallstones, islet cell tumor, primary brain tumor, acute and chronic lymphocytic and granulocytic tumors, hairy-cell tumor, adenoma, hyperplasia, medullary carcinoma, pheochromocytoma, mucosal neuron ms, intestinal ganglloneuromas, hyperplastic corneal nerve tumor, marfanoid habitus tumor, Wilm's tumor, seminoma, ovarian tumor, leiomyomater tumor, cervical dysplasia and in situ carcinoma, neuroblastoma, retinoblastoma, soft tissue sarcoma, malignant carcinoid, topical skin lesion, mycosis fungoide, rhabdomyosarcoma, Kaposi's sarcoma, osteogenic and other sarcoma, malignant hypercalcemia, renal cell tumor, polycythermia vera, adenocarcinoma, glioblastoma multiforma, leukemias, lymphomas, malignant melanomas, epidermoid carcinomas, and other carcinomas and sarcomas.

“Amino acid” is defined herein as any naturally occurring, artificial, or synthetic amino acid in either its L or D stereoisomeric forms, unless otherwise specified. The term “residue” is used interchangeably with the term “amino acid”, and is often designated as having a particular position in a given sequence of amino acids.

All amino acid abbreviations used in this disclosure are those accepted by the United States Patent and Trademark Office as set forth in 37 C.F.R. §1.822(b). The following one-letter amino acid designations are used in the description of the present invention. Xaa is used to designate an unknown or undesignated amino acid. The integers above specific residues of the structure provided herein define the residue position number.

“Amphiphilic” means the ability to dissolve in both water and lipids and/or having hydrophilic and lipophilic characteristics, and the terms “amphiphilic moiety” and “amphiphile” mean a moiety which is amphiphilic and/or which, when attached to a polypeptide or non-polypeptide drug, increases the amphiphilicity of the resulting conjugate, e.g., PEG-fatty acid oligomer, sugar fatty acid oligomer.

“Biologically active” refers to an agent having therapeutic or pharmacologic activity, such as an agonist, partial agonist or antagonist, among other actions. Non-limiting examples of such activity include neurogenesis enhancement, actions on neurite outgrowth, anti-inflammatory effects, apoptotic actions, myelin enhancement, neuroprotective properties, and the like.

“Hydrolyzable” refers to molecular bonds which are subject to hydrolysis.

“Hydrophilic” means the ability to dissolve in water, and the term “hydrophilic moiety” or “hydrophile” refers to a moiety which is hydrophilic and/or which when attached to another chemical entity, increases the hydrophilicity of such chemical entity. Examples include, but are not limited to, sugars and polyalkylene moieties such as polyethylene glycol.

“Lipophilic” means having an affinity for fat, such as chemicals that accumulate in fat and fatty tissues, the ability to dissolve in lipids and/or the ability to penetrate, interact with and/or traverse biological membranes, and the term, “lipophilic moiety” or “lipophile” means a moiety which is lipophilic and/or which, when attached to another chemical entity, increases the lipophilicity of such chemical entity.

“Alkyl” refers to a linear or cyclic monovalent hydrocarbon radical derived by the removal of one hydrogen atom from a single carbon atom of a parent alkane. The linear alkyl may be branched or straight-chain.

“Lower alkyl” refers to substituted or unsubstituted alkyl moieties having 1, 2, 3, 4, 5, or 6 carbon atoms.

“Alkenyl” refers to an unsaturated linear or cyclic alkyl radical having at least one carbon—carbon double bond derived by the removal of one hydrogen atom from a single carbon atom of a parent alkene. The linear alkenyl may be branched or straight-chain.

“Monodispersed” refers to a mixture of compounds wherein about 100 percent of the compounds in the mixture have the same molecular weight.

“Prodrug” refers to a biologically active agent that has been chemically derivitized such that, upon administration to a subject, the prodrug is converted to the biologically active agent.

“Polyalkylene glycol” or PAG refers to linear or branched polyalkylene glycol polymers such as polyethylene glycol (PEG), polypropylene glycol (PPG), and polybutylene glycol (PBG), and combinations thereof (e.g., linear or branched polymers including combinations of two or more different PAG subunits, such as two or more different PAG units selected from PEG, PPG, PPG, and PBG subunits), and includes the monoalkylether of the polyalkylene glycol. In a particular embodiment, the polyalkylene glycol is polyethylene glycol or “PEG.” The term “PEG subunit” refers to a single polyethylene glycol unit, i.e., —(CH₂CH₂O)—. The term “PPG subunit” refers to a single polypropylene glycol unit, i.e., —(CH₂CH₂CH₂O)—. The term “PBG subunit” refers to a single polybutylene glycol unit, i.e., —(CH₂CH₂CH₂CH₂O)—. PAG subunits may also include alkyl side chains, such as methyl, ethyl or propyl side chains.

The term “administering” or “administered” as used herein is meant to include any form of delivery described in the “pharmaceutical compositions” section below, including, but not limited to, parenteral and/or oral administration, as well as buccal, nasal, topical, intralesional, rectal, or ocular administration, among others, all of which are described in more detail in the “pharmaceutical compositions” section below. “Parenteral” is meant to include, but is not limited to, intravenous, subcutaneous, intramuscular, or transdermal administration. In the methods of the subject disclosure, the conjugated neuroactive steroids of the present disclosure may be administered alone, simultaneously with one or more other conjugated neuroactive steroid, or the compositions may be administered sequentially, in either order. It will be appreciated that the actual preferred method and order of administration will vary according to the particular preparation of conjugated neuroactive steroids being utilized, the particular formulation(s) of the one or more other conjugated neuroactive steroids being utilized. The optimal method and order of administration of the compositions of the disclosure for a given set of conditions can be ascertained by those skilled in the art using conventional techniques and in view of the information set out herein. In accordance with good clinical practice, it is preferred to administer the instant compositions at a concentration level which will produce effective beneficial effects without causing any harmful or untoward side effects.

According to the present disclosure, a “therapeutically effective amount” of a conjugated neuroactive steroid according to the present disclosure, or pharmaceutical composition thereof, is an amount which is sufficient for the desired pharmacological effect. As will be pointed out below, the exact amount required will vary from subject to subject, depending on age, general condition of the subject, the severity of the condition being treated, the particular biologically active agent administered, and the like. An appropriate “effective” amount in any individual case may be determined by one of ordinary skill in the art by reference to the pertinent texts and literature and/or by using routine experimentation.

The present disclosure provides methods for treating, ameliorating and/or preventing at least one physical symptom and/or at least one psychological symptom associated with, or resulting from, a phentotypic state of interest. As used herein, the term “symptom” refers to subjective or physical evidence of disease or physical disturbance observed by the subject. For example, representative symptoms of a phenotypic state of interest comprising a neurological or psychiatric disorder may include those set form in the DSM (e.g., DSM-IV, DSM-IN-TR, and/or the upcoming DSM-V), each of which is expressly incorporated herein by reference in its entirety. Such symptoms may be a physical symptom, a cognitive symptom, a psychological symptom, a negative symptom, or combinations thereof. Representative physical symptoms include, but are not limited to, dizziness, lightheadedness, chest/abdominal pain, nausea, increased heart rate/palpitations, headache, diarrhea, tremor, insomnia or other sleep disturbances, restlessness, weight gain, and appetite changes. Representative psychological symptoms include, but are not limited to, depression, irritability, agitation, aggression, difficulty concentrating, tension, anger, stress, delusions, paranoia, hallucinations, disorganization, indecision, and anxiety. Representative negative symptoms include, but are not limited to, affective flattening, alogia, and avolition. Representative cognitive symptoms include, but are not limited to, forgetfulness and memory problems, concentration difficulty, attentional problems, confusion, disorientation, dementia, delirium, symptoms related to learning disability, and symptoms related to mental retardation.

As used herein, the term “ameliorate” refers to the ability to make better, or more tolerable, a phenotypic state of interest. The term “prevent” refers to the ability to keep a phentotypic state of interest from happening or existing. The term “treating” refers to the caring for, or dealing with, a phentotypic state of interest either medically or surgically. Also within the scope of the term “treating” is the acting upon a subject with a phentotypic state of interest with a composition according to the present disclosure, to improve or alter the phenotypic state of interest.

The term “prophylaxis” and grammatical variations thereof are intended to refer to the prevention, inhibition, and/or lessening of the development of a symptom associated with neuropsychiatric disorder (NPD) in a subject whether that symptom is already present or not. As such, “prophylaxis” is not intended to refer only to modulating the development of a symptom in a subject in which the symptom is completely absent by is also intended to refer to ameliorating the symptom in a subject in which it exists as well as preventing, inhibiting, and/or lessening any worsening of the symptom in the subject that might occur for any reason. Thus, the term “prophylaxis” is intended to overlap with and yet be broader than the term “ameliorate.”

“Treat” or “treating” as used herein refers to any type of treatment that imparts a benefit to a subject afflicted with a disease or illness, including improvement in the condition of the subject (e.g., in one or more symptoms), delay in the progression of the condition, prevention or delay of the onset of the disease or illness, enhancement of normal physiological functionality, etc.

The present disclosure also provides methods for improving the cognitive functioning of a subject suffering from a phenotypic state of interest comprising a neurological or psychiatric disorder. As used herein, the term “improving cognitive functioning” refers to improving the cognitive functioning of a subject under any subjective or objective measure. One of ordinary skill in the art is aware of proper conditions under which to assess cognitive functioning, which can include various tests that are commonly employed. Representative, non-limiting tests include, but are not limited to, neuropsychological tests such as Continuous Performance Test (CPT), Wisconsin Card Sorting Test, Trailmaking A+B, the Mini Mental State Exam (MMSE), List Learning (Verbal Memory), Digit Sequencing Task (Working Memory), Token Motor Task (Motor Speed), Category Instances (Semantic Fluency), Controlled Oral Word Association Test (Letter Fluency), Tower of London Test (Executive Function), Symbol Coding (Attention and Motor Speed), Affective Interference Test-Delayed Recognition Task, Stroop Test, the Brief Assessment of Cognition in Schizophrenia (BACS; includes a number of the tests mentioned above), tests included in the Measurement and Treatment Research to Improve Cognition on Schizophrenia batters (MATRICS Consensus Cognitive Battery or MCCB), and the Alzheimer's Disease Assessment Scale-Cognitive Subscale (ADAS-cog).

The present disclosure further provides methods for the delaying or preventing the onset of, and/or decreasing the severity of, a symptom associated with a phenotypic state of interest. For example, in a subject suffering from a neurological or psychiatric disorder, a conjugated neuroactive steroid according to the present disclosure is administered as therapeutic to maintain a current state of well-being of the subject. Thus, in certain embodiments, the conjugated neuroactive steroid according to the present disclosure is administered to a subject as a maintenance therapy to prevent the worsening of symptoms that subjects afflicted with the disorder have and/or are at risk of developing.

In other embodiments, the subject does not have a phenotypic state of interest, but is at risk for developing one or more symptoms that are associated with a phenotypic state of interest, whether or not the subject develops a recognized phenotypic state of interest. The development of such symptoms can accompany the subject entering into a situation where stress, environmental conditions, genetic predispositions, anxiety, depression, and/or other hallmarks of a phenotypic state of interest can be elicited in an otherwise healthy subject. For example, for phenotypic states of interest comprising neurological or psychiatric disorders, such situations may include normal day-to-day activities that would be expected to cause stress, anxiety, and/or depression, but can also include extraordinary activities including, but not limited to, entry into combat or other life-threatening situations. The development of such symptoms can also occur as a result of other biochemical and/or biological alterations in the subject that are not caused by a neurological or psychiatric disorder, such as the, but not limited to, onset of menopause or completion of pregnancy (e.g., delivery of child).

### Compositions

Modified neuroactive steroids according to some embodiments of the present disclosure comprise, consist of, or consist essentially of a modified neuroactive steroid having the general formula R-Lₙ-NS, wherein R is —H, a modifying moiety, or a therapeutic agent, L is a linking group, n is an integer of about 0 to 50, wherein the integer represents the number of linking groups (also referred to herein as “linkers”), and NS is a neuroactive steroid. As used herein, the terms “neuroactive steroid” or “neurosteroid” are used interchangeably and refer to those steroids that are naturally occurring molecules synthesized in the brain or other areas of the body (or synthetic analogs thereof or derivatives thereof), many of which are capable of rapidly altering the excitability of neurons by binding to membrane-bound receptors, such as those for inhibitory and/or excitatory neurotransmitters. As used herein, the neuroactive steroids include those that demonstrate activity at membrane-bound ligand-gated ion channel receptors, such as GABA_{A} and NMDA receptors, among others. The terms “modified neuroactive steroid” and “conjugated neuroactive steroid” are used interchangeably and refer to neuroactive steroids that have been modified as described herein (e.g., by attachment of a modifying moiety and/or a therapeutic agent to the neuroactive steroid core, through modification of the neuroactive steroid core structure, etc.).

Examples of such neuroactive steroids include, but are not limited to, pregnenolone (PG); allopregnanolone (ALLO); epipregnanolone; epiallopregnanolone; androsterone; dehydroepiandrosterone (DHEA); progesterone; 3α-hydroxyprogesterone; 3β-hydroxyprogesterone; 5α-dihydroprogesterone; 5β-dihydroprogesterone; allotetrahydrodeoxycorticosterone (THDOC); cortisol-reduced and 11-deoxycortisol-reduced metabolites, such as 3α,5α-cortisol, 3α,5β-cortisol, 3α,5α-11-deoxycortisol, 3α,5β-11deoxycortisol, 5α-dihydrocortisol, and 5β-dihydrocortisol; and sulfated neurosteroids, such as pregnenolone sulfate, allopregnanolone sulfate, pregnanolone sulfate, dehydroepiandrosterone sulfate, among others; and pharmaceutically acceptable salts thereof, derivatives thereof, or combinations thereof. Particularly preferred neuroactive steroids include pregnenolone, allopregnanolone, dehydroepiandrosterone, progesterone, and pharmaceutically acceptable salts thereof, derivatives thereof, or combinations thereof.

For simplicity, the illustrations and descriptions provided below in sections 1-III utilize primarily pregnenolone as the neuroactive steroid component. However, it should be understood that any neuroactive steroid that comprises a hydroxyl group at the 3 position may also be modified as described herein, and are hence within the scope of the present disclosure.

I. Formulations of Neuroactive Steroid Core

One aspect of the present disclosure provides for a modified neuroactive steroid having the general formula R-Lₙ-NS, wherein R═H and n=0. In such embodiments, modifications are made to the core of the neuroactive steroid and/or the neuroactive steroid (or modified neuroactive steroid) is formulated to impart improved and/or enhanced pharmacologic effects, including, but not limited to, improved delivery (e.g., improved compliance from sustained or targeted delivery), extended release formulations, reduced metabolism and the like.

For example, for administration of a modified neuroactive steroid orally, the modified steroid may be formulated in a delayed or extended released manner. Alternatively, the crystalline size or polymorphic form of the neuroactive steroid component may be modulated, thereby slowing the dissolution and absorption of the modified neuroactive steroid. The formulations comprising the neuroactive steroids may be solutions, suspensions, liposomes, or nanoparticles. Such formulations may also employ targeting groups that provide the ability to target specific tissues, hence making the modified neuroactive steroids targeted or untargeted. Such modifications are described further in detail below and are well known to those skilled in the art (see, e.g., Patel et al. (2009) CNS Drugs 23:35-58; Packhaeuser, C. B. et al. (2004) Eur. J. Pharm. and Biopharm. 38:445-455; Shi, Y. et al., (2005) Expert Opin. Drug Deliv. 2:1039-1058; product insert Depo®-Tesosterone (Pharmacia & UpJohn Company)).

II. Compounds Based on Conjugation with the Neuroactive Steroid Core

Another aspect of the present disclosure provides for prodrug and/or poly-pharmacy formulations. The formulation approaches in section I may also be applied to any of the modified neuroactive steroids described herein.

### Direct Attachment

In certain embodiments, the modified neuroactive steroid comprises modifying moieties directly attached to the neuroactive steroid, and comprise the general formula R-Lₙ-NS, wherein R=a modifying moiety and n=0 (i.e., there is no L group). The modifying moieties may be used to impart improved and/or enhanced pharmacologic effects to the neuroactive steroid. For instance, modifying moieties may provide the neuroactive steroid with extended or sustained release properties, may facilitate targeting of the neuroactive steroid to specific cells and/or tissues of a subject, may enhance the pharmacologic effects of the neuroactive steroid, and/or may allow for the release of a molecular precursor of the neuroactive steroid, among other effects.

In one embodiment, the modifying moiety is positioned at C3 and/or C20 of the neuroactive steroid. An example of such a modified neuroactive steroid includes those having the following structure (Formula I):

wherein:

R₁ is —OH, a sulfate, a phosphate, or a modifying moiety;

R₂ is ═O, —OH, or a modifying moiety, and
denotes an optional C═C bond, with the proviso that there is not a C═C bond between both C17-C20 and C20-C21;

with the proviso that at least one of R₁ and R₂ is a modifying moiety.

In one embodiment, R₁ is —OH or a modifying moiety. In another embodiment, R₁ is the modifying moiety, and R₂ is ═O or —OH.

Exemplary steroids that may be modified in this manner include, but are not limited to, any of the following: pregnenolone, allopregnanolone, pregnanolone, epiallopregnanolone, epipregnanolone, pregnenolone sulfate, allopregnanolone sulfate, pregnanolone sulfate, and pharmaceutically acceptable salts thereof, derivatives thereof, or combinations thereof. Preferably, the neuroactive steroid is modified pregnenolone or modified allopregnanolone.

In one particular embodiment, the neuroactive steroid is pregnenolone, and the modified neuroactive steroid has the following structure:

wherein R is a modifying moiety.

Another example of a modified neuroactive steroid having a modifying moiety positioned at C3 and/or C20 includes those having the following structure (Formula II):

wherein:

R₁ is —OH, ═O, a sulfate, a phosphate, or a modifying moiety;

R₂ is ═O, —OH, or a modifying moiety, and
denotes an optional C═C bond, with the proviso that there is not a C═C bond between both C17-C20 and C20-C21;

with the proviso that at least one of R₁ and R₂ is a modifying moiety.

In one embodiment, R₁ is —OH, ═O, or a modifying moiety. In another embodiment, R₁ is the modifying moiety and R₂ is ═O or —OH.

Exemplary steroids that may be modified in this manner include, but are not limited to, any of the following: progesterone, 3α-hydroxyprogesterone, 3β-hydroxyprogesterone, 5α-dihydroprogesterone, 5β-dihydroprogesterone, and pharmaceutically acceptable salts thereof, derivatives thereof, or combinations thereof. Preferably, the neuroactive steroid is modified progesterone.

In another embodiment, the modifying moiety is positioned at C3 and/or C17 of the neuroactive steroid. An example of such a modified neuroactive steroid includes those having the following structure (Formula III):

wherein:

R₁ is —OH, a sulfate, a phosphate, or a modifying moiety;

R₂ is ═O or a modifying moiety; and
denotes an optional C═C bond;

with the proviso that at least one of R₁ and R₂ is a modifying moiety.

In one embodiment, R₁ is —OH or a modifying moiety. In another embodiment, R₁ is the modifying moiety and R₂ is ═O.

Exemplary steroids that may be modified in this manner include, but are not limited to, any of the following: androsterone, dehydroepiandrosterone, dehydroepiandrosterone sulfate, and pharmaceutically acceptable salts thereof, derivatives thereof, or combinations thereof. Preferably, the neuroactive steroid is modified dehydroepiandrosterone.

In still another embodiment, the modifying moiety may be positioned at C3, C11, C17, C20, and/or C21 of the neuroactive steroid. An example of such a modified neuroactive steroid includes those having the following structure (Formula IV):

wherein:

R₁ is ═O, —OH, a sulfate, a phosphate, or a modifying moiety,

R₂ is ═O, —OH, or a modifying moiety,

R₃ is —H, —OH, or a modifying moiety,

R₄, when present, is —H, —OH, or a modifying moiety, and

R₅ is —OH or a modifying moiety, and
denotes an optional C═C bond;

with the proviso that at least one of R₁, R₂, R₃, R₄, and R₅ is a modifying moiety.

In one embodiment, R₁ is ═O, —OH, or a modifying moiety. In another embodiment, R₁ is the modifying moiety, R₂ is ═O or —OH, R₃ is —H or —OH, R₄ is —H or —OH, and R₅ is —OH.

Exemplary steroids that may be modified in this manner include, but are not limited to, any of the following: allotetrahydrodeoxycorticosterone, and cortisol-reduced and 11-deoxycortisol-reduced metabolites, such as 3α,5α-cortisol, 3α,5β-cortisol, 3α,5α-11-deoxycortisol, 3α,5β-deoxycortisol, 5α-dihydrocortisol, 5β-dihydrocortisol, and pharmaceutically acceptable salts thereof, derivatives thereof, or combinations thereof.

The modifying moiety may be a moiety that imparts improved and/or enhanced pharmacologic effects to the neuroactive steroid. Selection of an appropriate modifying moiety will depend on the desired effect. For instance, some modifying moieties may alter the delivery rate of the neuroactive steroid, for example, by providing the neuroactive steroid with extended or sustained release properties, altering the rate of breakdown and/or absorption of the neuroactive steroid, and/or altering the crystallinity of the neuroactive steroid. Other modifying moieties may facilitate targeting of the neuroactive steroid to specific cells and/or tissues of a subject, may enhance the pharmacologic effects of the neuroactive steroid, and/or may allow for the release of a molecular precursor of the neuroactive steroid, among other effects.

In one particular embodiment, the modifying moiety is a hydrophobic ester of a carboxylic acid. Such esters may be used to produce modified neuroactive steroids that have sustained release properties. Examples of such esters include, but are not limited to, cypionate, succinate, disuccinate, and substituted or unsubstituted, branched or straight-chain C₃-C₂₄ carboxylic acid esters (including C₇-C₁₂ carboxylic acid esters as well as C₃-C₂₄ diesters), such as pentanoate, heptanoate, octanoate, nonanoate, decanoate, dodecanoate, 2-propylpentanoate, and the like, and combinations thereof. The carboxylic acid esters may be substituted at one or more position with —OH, —NH₂, or —F. In one embodiment, the modifying moiety is selected from the group consisting of pentanoate, heptanoate, octanoate, nonanoate, decanoate, dodecanoate, cypionate, succinate, disuccinate, 2-propylpentanoate, and combinations thereof.

Specific examples of neuroactive steroids modified with carboxylic acid esters include, but are not limited to, pregnenolone pentanoate, pregnenolone heptanoate, pregnenolone octanoate, pregnenolone nonanoate, pregnenolone decanoate, pregnenolone dodecanoate, pregnenolone 2-propylpentanoate, pregnenolone cypionate, pregnenolone succinate, pregnenolone disuccinate, and pharmaceutically acceptable salts, thereof, and derivatives thereof. Other specific examples of such modified neuroactive steroids include, but are not limited to, allopregnanolone pentanoate, allopregnanolone heptanoate, allopregnanolone octanoate, allopregnanolone nonanoate, allopregnanolone decanoate, allopregnanolone dodecanoate, allopregnanolone 2-propylpentanoate, allopregnanolone cypionate, allopregnanolone succinate, allopregnanolone disuccinate, and pharmaceutically acceptable salts thereof, and derivatives thereof. Other specific examples of modified neuroactive steroids include, but are not limited to, dehydroepiandrosterone pentanoate, dehydroepiandrosterone heptanoate, dehydroepiandrosterone octanoate, dehydroepiandrosterone nonanoate, dehydroepiandrosterone decanoate, dehydroepiandrosterone dodecanoate, dehydroepiandrosterone 2-propylpentanoate, dehydroepiandrosterone cypionate, dehydroepiandrosterone succinate, dehydroepiandrosterone disuccinate, and pharmaceutically acceptable salts thereof, and derivatives thereof.

In some embodiments, the carbonyl carbon of the carboxylic acid ester may be substituted with a heteroatom selected from the group consisting of sulfur and phosphorus, and the modifying moiety may be a phosphonate or sulfonate. Examples of such modifying moieties include those having the following structure:

wherein X₁ is S or P; R″ is a substituted or unsubstituted, linear or cyclic alkyl, a substituted or unsubstituted, linear or cyclic alkenyl, or a polyalkylene glycol; and R′ is —OH or ═O. The polyalkylene glycol may be, for example, a polyethylene glycol. The alkyl and the alkenyl may be substituted at one or more position with —OH, —NH₂, or —F. Typically, R″ is a substituted or unsubstituted C₃-C₂₄ alkyl (including C₇-C₁₂ alkyl) or a substituted or unsubstituted C₃-C₂₄ alkenyl (including C₇-C₁₂ alkenyl). In one embodiment, the modifying moiety is selected from the group consisting of a C₇-C₁₂ alkyl phosphonate and a C₇-C₁₂ alkyl sulfonate. In another embodiment, the modifying moiety is selected from the group consisting of:

In another embodiment, the modifying moiety is a phosphonate or sulfonate selected from the group consisting of

In other embodiments, the modifying moiety may be a phosphate or sulfate ester. Examples of such esters include those having the following structure:

wherein X₁ is S or P; R″ is a substituted or unsubstituted, linear or cyclic alkyl, a substituted or unsubstituted, linear or cyclic alkenyl, or a polyalkylene glycol; and R′″ is —OH or ═O. The polyalkylene glycol may be, for example, a polyethylene glycol. The alkyl and alkenyl may be substituted at one or more position with —OH, —NH₂, or —F. R″ may be, for example, a C₃-C₂₄ alkyl (including C₇-C₁₂ alkyl), or a C₃-C₂₄ alkenyl (including C₇-C₁₂ alkenyl). Specific examples of such esters include, but are not limited to, substituted or unsubstituted C₃-C₂₄ (including substituted or unsubstituted C₇-C₁₂) phosphate esters, such as C₃-C₂₄ alkyl phosphates and C₃-C₂₄ alkenyl phosphates, substituted or unsubstituted C₃-C₂₄ (including substituted or unsubstituted C₇-C₁₂) sulfate esters, such as C₃-C₂₄ alkyl sulfates and C₃-C₂₄ alkenyl sulfates, and a cypionate wherein the carbonyl is substituted with a phosphate or sulfate (referred to herein as a phosphate or sulfate substituted cypionate). In one particular embodiment, the modifying moiety has a structure selected from the group
consisting of

In another embodiment, the modifying moiety is a succinate wherein at least one of the carbonyls is substituted with a phosphate or sulfate (referred to herein as a phosphate or sulfate substituted succinate), or a disuccinate wherein at least one of the carbonyls is substituted with a phosphate or sulfate (referred to herein as a phosphate or sulfate substituted disuccinate).

In one particular embodiment, the modifying moiety is a phosphate or sulfate ester selected from the group consisting of

Thus, in one embodiment, the modifying moiety is selected from the group consisting of substituted or unsubstituted C₃-C₂₄ (including C₇-C₁₂) phosphate esters, C₃-C₂₄ (including C₇-C₁₂) alkyl phosphonate, phosphate substituted cypionate, phosphate substituted succinate, phosphate substituted disuccinate, and combinations thereof.

In another embodiment, the modifying moiety is selected from the group consisting of substituted or unsubstituted C₃-C₂₄ (including C₇-C₁₂) sulfate esters, C₃-C₂₄ (including C₇-C₁₂) alkyl sulfonate, sulfate substituted cypionate, sulfate substituted succinate, sulfate substituted disuccinate, and combinations thereof.

In still other embodiments, the modifying moiety is an enol derivative of any of the above-described esters. Such a modification would allow the release of the neuroactive steroid by a hydrolytic mechanism, either alone or as a result of being catalyzed by an enzyme or other biological molecule. Non-limiting examples of such modifying moieties include the enol ester of cypionate, the enol ester of heptanoate, the enol ester of decanoate, the enol ester of dodecanoate, and the like, and combinations thereof.

In other embodiments, the modifying moiety is —OR, wherein R═X number of carbon atoms, and X is an integer between 0 and 100. The R group may optionally be unsaturated. In one embodiment, the modifying moiety is —OR, and R is a substituted or unsubstituted C₁-C₉₉ alkyl or a substituted or unsubstituted C₁-C₉₉ alkenyl. The alkyl and alkenyl may be substituted at one or more position with —OH, —NH₂, or —F. In one specific embodiment, the modifying moiety is —OR, and R is a C₁-C₂₄ alkyl or a C₁-C₂₄ alkenyl. In certain embodiments, the modifying moiety comprises an unsaturated fatty acid. Non-limiting examples of suitable unsaturated fatty acids include omega-3 fatty acids, omega-6 fatty acids, omega-9 fatty acids, and combinations thereof.

In other embodiments, the modified neuroactive steroid may be designed to target specific cells/tissues and/or enhance the pharmacologic effects of the neuroactive steroid. For example, the modifying moiety may comprise a chemical delivery system (CDS). In such embodiments, the modified neuroactive steroid may have a structure as set forth in any one of Formulas I-IV, wherein the modifying moiety is:

wherein X₂ is C, P, or S; R″ is a substituted or unsubstituted, linear or cyclic alkyl, a substituted or unsubstituted, linear or cyclic alkenyl, or a polyalkylene glycol; m is an integer from 0 to 50, t is an integer of 0 or 1; R′ is a CDS group, and R′″, when present, is —OH or ═O. The polyalkylene glycol may be, for example, a polyethylene glycol. The alkyl and alkenyl may be substituted at one or more position with —OH, —NH₂, or —F. In one embodiment, m is an integer from 1 to 24. In some embodiments, the modifying moiety is attached to a neuroactive steroid at the C3 position. An example of such a structure, where t═O, m═O, X₂═C, and R′″ is not present, using pregnenolone as the neuroactive steroid would be as follows:

wherein R′ is a chemical delivery system group.

As used herein, the term “chemical delivery system” refers to any molecule which can be attached to a neuroactive steroid and facilitate the delivery of the neuroactive steroid to the desired cell type and/or tissue of a subject. Such molecules include, but are not limited to, nicotinic acid ester derivatives (CNS targeting properties), amino acids, peptides, proteins and the like. Many such molecules are well known in the art and will be dependent on the desired tissue/cell type to be targeted. In preferred embodiments, the targeting molecule is one that facilitates delivery of the modified neuroactive steroid across the blood/brain barrier, such as dihydronicotinic acid, and derivatives thereof (see, e.g., Rautio, J. et al. (2008) AAPS Journal 10:92-102; Patel, M. M. et al. (2009) CNS Drugs 23:35-58).

In one particular embodiment, the modified neuroactive steroid has the structure set forth in Formula I, where the modifying moiety is selected from the group consisting of cypionate, disuccinate, substituted or unsubstituted, branched or straight-chain C₃-C₂₄ carboxylic acid esters (including C₇-C₁₂ carboxylic acid esters),

a phosphate substituted succinate, a sulfate substituted succinate, a phosphate substituted disuccinate, a sulfate substituted disuccinate, an enol ester derivative, —OR,

and combinations thereof, wherein R is a substituted or unsubstituted C₁-C₉₉ alkyl or a substituted or unsubstituted C₁-C₉₉ alkenyl; X₁ is S or P; X₂ is C, P, or S; R″ is a substituted or unsubstituted, linear or cyclic alkyl, a substituted or unsubstituted, linear or cyclic alkenyl, or a polyalkylene glycol; m is an integer from 0 to 50; t is an integer of 0 or 1; R′ is a CDS group; and R′″, when present, is —OH or ═O. In one particular embodiment, the modified neuroactive steroid has the structure set forth in Formula I, and the modifying moiety is selected from the group consisting of cypionate, succinate, disuccinate, pentanoate, heptanoate, octanoate, nonanoate, decanoate, dodecanoate, 2-propylpentanoate, and combinations thereof. In another particular embodiment, the neuroactive steroid has the structure set forth in Formula I, and the modifying moiety is selected from the group consisting of cypionate, heptanoate, decanoate, 2-propylpentanoate, and combinations thereof.

In another embodiment, the modified neuroactive steroid has the structure set forth in Formula II, where the modifying moiety is selected from the group consisting of cypionate, succinate, disuccinate, substituted or unsubstituted, branched or straight-chain C₃-C₂₋₄ carboxylic acid esters (including C₇-C₁₂ carboxylic acid esters),

a phosphate substituted succinate, a sulfate substituted succinate, a phosphate substituted disuccinate, a sulfate substituted disuccinate, an enol ester derivative, —OR,

and combinations thereof, wherein R is a substituted or unsubstituted C₁-C₉₉ alkyl or a substituted or unsubstituted C₁-C₉₉ alkenyl; X₁ is S or P; X₂ is C, P, or S; R″ is a substituted or unsubstituted, linear or cyclic alkyl, a substituted or unsubstituted, linear or cylcic alkenyl, or a polyalkylene glycol; m is an integer from 0 to 50; t is an integer of 0 or 1; R′ is a CDS group, and R′″, when present, is —OH or ═O. In one particular embodiment, the modified neuroactive steroid has the structure set forth in Formula II, and the modifying moiety is selected from the group consisting of cypionate, succinate, disuccinate, pentanoate, heptanoate, octanoate, nonanoate, decanoate, dodecanoate, 2-propylpentanoate, and combinations thereof. In another particular embodiment, the neuroactive steroid has the structure set forth in Formula II, and the modifying moiety is selected from the group consisting of cypionate, heptanoate, decanoate, 2-propylpentanoate, and combinations thereof.

In another embodiment, the modified neuroactive steroid has the structure set forth in Formula III, where the modifying moiety is selected from the group consisting of succinate, disuccinate, substituted or unsubstituted branched or straight-chain C₃-C₂₄ carboxylic acid esters (including C₇-C₁₂ carboxylic acid esters),

a phosphate substituted succinate, a sulfate substituted succinate, a phosphate substituted disuccinate, a sulfate substituted disuccinate, an enol ester derivative, —OR,

and combinations thereof, wherein R is a substituted or unsubstituted C₁-C₉₉ alkyl or a substituted or unsubstituted C₁-C₉₉ alkenyl; X₁ is S or P; X₂ is C, P, or S; R″ is a substituted or unsubstituted, linear or cyclic alkyl, a substituted or unsubstituted, linear or cyclic alkenyl, or a polyalkylene glycol; m is an integer from 0 to 50; t is an integer of 0 or 1; R′ is a CDS group, and R′″, when present, is —OH or ═O. In one particular embodiment, the modified neuroactive steroid has the structure set forth in Formula III, and the modifying moiety is selected from the group consisting of cypionate, succinate, disuccinate, pentanoate, heptanoate, octanoate, nonanoate, decanoate, dodecanoate, 2-propylpentanoate, and combinations thereof. In another particular embodiment, the neuroactive steroid has the structure set forth in Formula III, and the modifying moiety is selected from the group consisting of cypionate, heptanoate, decanoate, 2-propylpentanoate, and combinations thereof.

In another embodiment, the modified neuroactive steroid has the structure set forth in Formula IV, where the modifying moiety is selected from the group consisting of cypionate, succinate, disuccinate, substituted or unsubstituted, branched or straight-chain C₃-C₂₋₄ carboxylic acid esters (including C₇-C₁₂ carboxylic acid esters).

a phosphate substituted succinate, a sulfate substituted succinate, a phosphate substituted disuccinate, a sulfate substituted disuccinate, an enol ester derivative, —OR,

and combinations thereof, wherein R is a substituted or unsubstituted C₁-C₉₉ alkyl or a substituted or unsubstituted C₁-C₉₉ alkenyl; X₁ is S or P; X₂ is C, P, or S; R″ is a substituted or unsubstituted, linear or cyclic alkyl, a substituted or unsubstituted, linear or cyclic alkenyl, or a polyalkylene glycol; m is an integer from 0 to 50; t is an integer of 0 or 1; R′ is a CDS group, and R′″, when present, is —OH or ═O. In one particular embodiment, the modified neuroactive steroid has the structure set forth in Formula IV, and the modifying moiety is selected from the group consisting of cypionate, succinate, disuccinate, pentanoate, heptanoate, octanoate, nonanoate, decanoate, dodecanoate, 2-propylpentanoate, and combinations thereof. In another particular embodiment, the neuroactive steroid has the structure set forth in Formula IV, and the modifying moiety is selected from the group consisting of cypionate, heptanoate, decanoate, 2-propylpentanoate, and combinations thereof.

Specific examples of modified neuroactive steroids are set forth in Table 1. For simplicity, the illustrations and descriptions provided below utilize pregnenolone as the neuroactive steroid component. However, it should be understood that similar modifications can be made to any neuroactive steroid that comprises a hydroxyl group or carbonyl at any of the positions indicated in Formulas I-IV above, and such modified neuroactive steroids are hence within the scope of the present disclosure. Similarly, the illustrations and descriptions provided below utilize carboxylic acid esters or enol derivatives as the modifying moiety. However, it should be understood that modifications where the carbonyl of the carboxylic acid ester is substituted with a phosphate or sulfate are also within the scope of the present disclosure.

```
TABLE 1
Exemplary Compounds showing the Direct Attachment of Moieties to a
Neuroactive Steroid
  Compound A
  Compound B
  Compound C
  Compound D
  Compound E
  Compound F
  Compound G
```

In one embodiment, the modifying moiety may be attached to the hydroxyl group at the C3 position, as shown in Compounds A, F, and G of Table 1 above.

In another embodiment, the modifying moiety is an enol ester derivative attached to C20 of the neuroactive steroid (see Compound B of Table 1).

In another embodiment, modified neuroactive steroids may be created to allow the release a molecular precursor of the neuroactive steroid. For example, an ester derivative may be positioned at C3 of the neuroactive steroid and a hydroxyl group at C20 (see, e.g., Compound C of Table I). Alternatively, a hydroxyl group may be positioned at C3 and an ester derivative positioned at C20 of the neuroactive steroid (see, e.g., Compound D of Table 1). Further, a modification may include an ester derivative positioned at both C3 and C20 (see, e.g., Compound E of Table 1).

In still another embodiment, specific examples of modified neuroactive steroids are selected from the following structures:

In still another embodiment, specific examples of modified neuroactive steroids are selected from the following structures:

### Attachment Via Linker

In yet another embodiment, the modified neuroactive steroid is combined with the same, or different neuroactive steroid, or other groups via a linker. Modified neuroactive steroids according to these embodiments comprise the following general formula: R-Lₙ-NS, wherein R=a neuroactive steroid or other group as described herein, L=linker, n=an integer that is equal to, or greater than, 1, and NS=a neuroactive steroid. In one example, a neuroactive steroid is coupled through a linker, such as a carbonyl group, to another molecule of the same or different neuroactive steroid, sustained releasing group, a targeting group, a pharmacologically active group, and the like. In certain embodiments, a spacer may be included within the linker in order to modify or tune the rate of neuroactive steroid release.

For example, the modified neuroactive steroids of the present disclosure may comprise from 1 to 50 linker molecules (e.g., n is 1 to 50), from 1 to 40 linker molecules (e.g., n is 1 to 40), from 1 to 30 linker molecules (e.g., n is 1 to 30), from 1 to 20 linker molecules (e.g., n is 1 to 20), or from 1 to 10 linker molecules (e.g., n is 1 to 10) attached together.

As used herein, the terms “linker” or “linking group,” used interchangeably herein, refer to any agent or molecule that bridges the neuroactive steroid to the R moiety. One of ordinary skill in the art will recognize that sites on the neuroactive steroid that are not necessary for the function of the neuroactive steroid are ideal sites for attaching a linker and/or a targeting moiety (e.g., a CDS or other moiety suitable for targeting the neuroactive steroid to the desired tissues in a subject), provided that the linker and/or targeting moiety, once attached to the neuroactive steroid, do(es) not interfere with the function of the neuroactive steroid. Some examples of suitable positions on the neuroactive steroid to which the linkers may attach include, but are not limited to, the C3, C11, C17, C20, and/or C21 position (such as set forth in Formulas I-IV) or at any other position on the neuroactive steroid having an —OH or a ═O substituent. Preferably, the linker is attached to the neuroactive steroid at a position having an —OH substituent. In one preferred embodiment, the linker is attached to the neuroactive steroid at the C3 position.

Some examples of suitable linkers for use in the present disclosure include, but are not limited to, those set forth in Table 2 below.

```
TABLE 2
Exemplary Linkers
examples of linkers
L =

  w = 0-20
  cis or trans
  m = 2-20
  m = 2-20
  m = 2-20 t = 1-20 U = —O—(C═O)—R', = —NH—(C═O)—R', = —NR*—(C═O)—R' = —OH, or = —NH₂ V = H, CH₃



n = 1
```

D is —H, —CH₃, —CO₂H, —CO₂R′, —CH₂CO₂H, or —CH₂CO₂R′; R′ is independently selected from NS (i.e., a neuroactive steroid), R (i.e., H, a therapeutic agent, a modifying moiety, or another neuroactive steroid) an alkyl (e.g., a lower alkyl), and another linker, and R* is —H or an alkyl (e.g., a lower alkyl). In some embodiments, w may be 1-20, including 1-10 or 1-5.

In some embodiments, the linkers can be further modified by, for example, placing nitrogen substituents along the chain.

The linkers may be attached to the neuroactive steroid using any suitable means known to those skilled in the art. Typically, one or more of the carbonyl groups in the linker will form an ester or carbonate ester linkage with an —OH or ═O substituent on the neuroactive steroid. One skilled in the art would readily understand how to form such linkages using the linkers set forth in Table 2.

In embodiments where the linker is used to link the neuroactive steroid to another compound, e.g., where R is selected from another neuroactive steroid, a therapeutic agent, or a modifying agent as discussed hereinafter, one or more of the carbonyl groups in the linker will form an ester or carbonate ester linkage with an —OH or ═O substituent on the R moiety (e.g., on the neuroactive steroid, therapeutic agent, or modifying moiety). The carbonyl group that forms this linkage may be the same or different carbonyl group as used to attach the linker to the neuroactive steroid. Similarly, in embodiments where n>1 (i.e., where there is more than one linker), one or more of the carbonyl groups in the linker may form an ester or carbonate ester linkage with a ═O substituent on another linker. Some non-limiting examples of suitable linkages are illustrated below.

For instance, in one embodiment, the linker

is used link a neuroactive steroid to an R moiety, where R is selected from the group consisting of a neuroactive steroid, a therapeutic agent, and a modifying moiety. The neuroactive steroid may be linked to the R moiety by way of a carbonate ester linkage, illustrated as follows:

Alternately, the linker is

where w=0 (i.e., the linker is

and the neuroactive steroid is linked to the R moiety by way of an oxalate linkage, illustrated as follows:

In another embodiment, the linker is

where w=1-20, and the neuroactive steroid is linked to the R moiety by way of an ester linkage, illustrated as follows:

Some other non-limiting examples of suitable positions where the linkers can be attached to the neuroactive steroid (or R moiety) are illustrated in Table 2 with an R′ moiety. Depending on the desired linkage, R′ can be selected from R (i.e., a neuroactive steroid, H, a therapeutic agent, or a modifying moiety), an alkyl, or another linker. It should be understood that positions on the linkers other than those designated with an R′ moiety could also be used to attach the linker to the neuroactive steroid and/or R moiety, such as, for example, other carbonyl groups present in the linkers.

Thus, in one embodiment, the modified neuroactive steroid has the general formula R-Lₙ-NS, wherein R is selected from a neuroactive steroid, H, a therapeutic agent, a modifying moiety, and combinations thereof; L is a linker selected from the linkers set forth in Table 2; n is an integer equal to, or greater than, 1; and NS is a neuroactive steroid. Preferably, n is an integer of from 1 to 50, including 1 to 40, 1 to 30, 1 to 20, and 1 to 10. It should be understood that in embodiments where the R moiety is a neuroactive steroid, the R moiety neuroactive steroid may be either the same neuroactive steroid or a different neuroactive steroid as that represented by the NS moiety. Furthermore, for purposes of this embodiment, the neuroactive steroid may be a naturally occurring neuroactive steroid and/or may be a neuroactive steroid that has been modified as described herein (e.g., via direct attachment of a modifying moiety as described above and/or via modification of the neuroactive steroid as described herein in sections I or III).

Examples of such modified neuroactive steroids include those having the following structures (Formulas V-VII):

wherein R is selected from a neuroactive steroid, H, a therapeutic agent, a modifying moiety, and combinations thereof; L is a linker selected from the linkers set forth in Table 2; n is an integer equal to, or greater than, 1; R₂ is ═O or —OH; R₃ is —H or —OH; R₄ is —H or —OH, and ----- is an optional C═C bond. Preferably, n is an integer from 1 to 50. In embodiments where the modified neuroactive steroid has the structure of Formula V, there is not a C═C bond between both the C4-C5 and C5-C6 positions.

Exemplary neuroactive steroids that may be modified in accordance with Formula V include, but are not limited to, any of the following: pregnenolone, allopregnanolone, epiallopregnanolone, epipregnanolone, progesterone, 3α-hydroxyprogesterone, 3β-hydroxyprogesterone, 5α-dihydroprogesterone, 5β-dihydroprogesterone, and the like. Exemplary neuroactive steroids that may be modified in accordance with Formula VI include, but are not limited to, any of the following: androsterone, dehydroepiandrosterone, and the like. Exemplary neuroactive steroids that may be modified in accordance with Formula VII include, but are not limited to, any of the following: allotetrahydrodeoxycorticosterone, and cortisol-reduced and 11-deoxycortisol-reduced metabolites, such as 3α,5α-cortisol, 3α,5β-cortisol, 3α,5α-11-deoxycortisol, 3α,5β-11-deoxycortisol, 5α-dihydrocortisol, 5β-dihydrocortisol, and the like.

In another embodiment, the linker may comprise one or more sulfur or phosphorus atom and may be a sulfonate or phosphonate. Some examples of suitable sulfonate or phosphonate linkers for use in the present disclosure include, but are not limited to, those set forth in Table 3 below.

```
TABLE 3
Exemplary Sulfonate and Phosphonate Linkers
examples of linkers
L =
  w = 0-20
  cis or trans
  m = 2-20
  m = 2-20 t = 1-20 U = —O—X(O)ᵤ—(OyR')ₛ, = —NH—X(O)ᵤ—(OyR')ₛ, = —NR*—X(O)ᵤ—(OyR')ₛ, = —OH, or = —NH₂ V = H, CH₃
  m = 2-20


n = 1
```

For the linkers in Table 3, X is independently selected from C, S, and P, with the proviso that at least one X in each linker is S or P. R₅, when present, is independently selected from ═O and —OR₇; R₆ is independently selected from —H and —OR₇; R₇ is —H or R′; u is 1 or 2; s is 1 or 2; y is 0 or 1; R* is —H or an alkyl (e.g., a lower alkyl); and R′ is independently selected from NS (i.e., a neuroactive steroid), R (i.e., —H, a therapeutic agent, a modifying moiety, or another neuroactive steroid), an alkyl (e.g., a lower alkyl), and another linker.

In some embodiments, the linkers can be further modified by, for example, placing nitrogen substituents along the chain.

In another embodiment, the linker may comprise one or more sulfur or phosphorus atom and may be a sulfate or phosphate derivative. Some examples of suitable sulfate or phosphate linkers for use in the present disclosure include, but are not limited to, those set forth in Table 4 below.

```
TABLE 4
Exemplary Sulfate and Phosphate Linkers
examples of linkers
L =
  w = 0-20
  m = 2-20
  m = 2-20 t = 1-20 U = —O—X(O)ᵤ—(OyR')ₛ, = —NH—X(O)ᵤ—(OyR')ₛ, = —NR*—X(O)ᵤ—(OyR')ₛ, = —OH, or = —NH₂ V = H, CH₃
  m = 2-20



n = 1
```

For the linkers in Table 4, X is independently selected from C, S, and P, with the proviso that at least one X in each linker is S or P. D is —H, —CH₃, —CO₂H, —CO₂R′, —CH₂CO₂H, or —CH₂CO₂R′; R₅, when present, is independently selected from ═O and —OR₇; R₆ is independently selected from —H and —OR₇; R₇ is —H or R′; u is 1 or 2; s is 1 or 2; y is 0 or 1; z is 0 or 1; R* is —H or an alkyl (e.g., a lower alkyl); and R′ is independently selected from NS (i.e., a neuroactive steroid), R (i.e., —H, a therapeutic agent, a modifying moiety, or another neuroactive steroid), an alkyl (e.g., a lower alkyl), and another linker.

In some embodiments, the linkers can be further modified by, for example, placing nitrogen substituents along the chain.

The sulfur and phosphorus containing linkers may be attached to the neuroactive steroid using any suitable means known to those skilled in the art. In one embodiment, the linkers may be attached to the neuroactive steroid at one or more carbonyl group present in the linker (e.g., where X is C). For instance, one or more of the carbonyl groups in the linker may form an ester or carbonate ester linkage with an —OH or ═O substituent on the neuroactive steroid. In embodiments where the linker is used to link the neuroactive steroid to another compound, e.g., where R is selected from another neuroactive steroid, a therapeutic agent, or a modifying agent as discussed herein, one or more of the carbonyl groups in the linker may form an ester or carbonate ester linkage with an —OH or ═O substituent on the R moiety (e.g., on the neuroactive steroid, therapeutic agent, or modifying agent). Similarly, in embodiments where n>1 (i.e., where there is more than one linker), one or more of the carbonyl groups in the linker may form an ester or carbonate ester linkage with a ═O substituent on another linker. The carbonyl group that forms these linkages may be the same or different carbonyl group as used to attach the linker to the neuroactive steroid. Examples of such linkages are described above. One skilled in the art would readily understand how to form such linkages using the linkers set forth in Tables 3 and 4.

Alternately or in addition, the sulfur and/or phosphate containing linkers may be attached to the neuroactive steroid (and/or to the R moiety) using one or more of the phosphorus or sulfurs in the linker (e.g., where X is S or P). For instance, one or more of the phosphorus or sulfur atoms in the linker may form a sulfate or phosphate linkage with an —OH or ═O substituent on the neuroactive steroid or, in embodiments where the linker is used to link the neuroactive steroid to another compound (e.g., where R is selected from another neuroactive steroid, a therapeutic agent, or a modifying moiety as discussed herein), one or more of the phosphorus or sulfur atoms in the linker may form a phosphate or sulfate linkage with an —OH substituent on the R moiety Similarly, in embodiments where n>1 (i.e., where there is more than one linker), one or more of the phosphorus or sulfur atoms in the linker may form a phosphate or sulfate linkage with a ═O or —OH substituent on another linker. The sulfur or phosphorus that forms a linkage with the R moiety may be the same or different sulfur or phosphorus used to attach the linker to the neuroactive steroid.

In some embodiments, linkages can be created using both a carbonyl group and a sulfur and/or phosphorus present in the linker. For instance, one or more sulfur and/or phosphorus in the linker may form a linkage with an —OH substituent on the neuroactive steroid, while one or more carbonyl group in the linker may form an ester or carbonate ester linkage with an —OH substituent on the R moiety (e.g., the neuroactive steroid, therapeutic agent, or modifying moiety). Alternately, one or more sulfur and/or phosphorus in the linker may form a linkage with an —OH substituent on the R moiety, while one or more carbonyl group in the linker may form an ester or carbonate ester linkage with an —OH or ═O substituent on the neuroactive steroid.

Some non-limiting examples of suitable linkages using phosphorus or sulfur containing linkers are set forth below.

In one embodiment, if the linker is

where w=1-20, one X is S or P and the other X is C, and z, R₅, and R₆ are as defined above for the linkers in Table 4, the neuroactive steroid may be linked to the R moiety using the phosphate or sulfate and the carbonyl group, illustrated as follows:

In another embodiment, if the linker is

where w=1-20, one X is S or P and the other X is C, and R₅ and R₆ are as defined above for the linkers in Table 3, the neuroactive steroid may be linked to the R moiety using the phosphate or sulfate and the carbonyl group, illustrated as follows:

Some other non-limiting examples of suitable positions where the linkers can be attached to the neuroactive steroid (or R moiety or another linker) are illustrated in Tables 3 and 4 with an R′ moiety. Depending on the desired linkage, R′ can be either R (i.e., a neuroactive steroid, H, a therapeutic agent, or a modifying moiety), an alkyl, or another linker. It should be understood that positions on the linkers other than those designated with an R′ moiety could also be used to attach the linker to the neuroactive steroid, such as, for example, other carbonyl groups or any P or S present in the linkers.

Thus, in one embodiment, the modified neuroactive steroid has the general formula R-Lₙ-NS, wherein R is selected from a neuroactive steroid, H, a therapeutic agent, a modifying moiety, and combinations thereof; L is a sulfur- or phosphorus-containing linker (e.g., a sulfate, phosphate, sulfonate, or phosphonate linker) selected from the linkers set forth in Tables 3 and 4; n is an integer equal to, or greater than, 1; and NS is a neuroactive steroid. Preferably, n is an integer of from 1 to 50. It should be understood that in embodiments where the R moiety is a neuroactive steroid, the R moiety neuroactive steroid may be either the same neuroactive steroid or a different neuroactive steroid as that represented by the NS moiety. Furthermore, for purposes of this embodiment, the neuroactive steroid may be a naturally occurring neuroactive steroid and/or may be a neuroactive steroid that has been modified as described herein (e.g., via direct attachment of a modifying moiety as described above and/or via modification of the neuroactive steroid as described herein in sections I or III).

Examples of such modified neuroactive steroids include those having the structures set forth in Formulas V-VII, wherein R is selected from a neuroactive steroid, H, a therapeutic agent, a modifying moiety, and combinations thereof; L is a sulfur- or phosphorus-containing linker selected from the linkers set forth in Tables 3 and 4; n is an integer equal to, or greater than, 1; R₂ is ═O or —OH; R₃ is —H or —OH; R₄ is —H or —OH, and is an optional C═C bond. Preferably, n is an integer from 1 to 50.

In another embodiment, the modified neuroactive steroid has the following structure:

wherein R is selected from a neuroactive steroid, H, a therapeutic agent, a modifying moiety, and combinations thereof; L is a sulfur- or phosphorus-containing linker selected from the linkers set forth in Tables 3 and 4; n is an integer equal to, or greater than, 1; R₁ is ═O or —OH; R₂ is ═O or —OH; R₃ is —H or —OH; and R₄ is —H or —OH. Preferably, n is an integer from 1 to 50.

In other embodiments, the modified neuroactive steroid has the general formula R-Lₙ-NS, wherein R is selected from a neuroactive steroid, a therapeutic agent, a modifying moiety, and combinations thereof; L is a sulfur- or phosphorus-containing linker; n is an integer equal to, or greater than, 1; and NS is a neuroactive steroid. Preferably, n is an integer of from 1 to 50. In embodiments where the R moiety is a neuroactive steroid, the R moiety neuroactive steroid may be either the same neuroactive steroid or a different neuroactive steroid as that represented by the NS moiety. Furthermore, for purposes of this embodiment, the neuroactive steroid may be a naturally occurring neuroactive steroid and/or may be a neuroactive steroid that has been modified as described herein (e.g., via direct attachment of a modifying moiety as described above and/or via modification of the neuroactive steroid as described herein in sections I or III). In one embodiment, the linker is selected from the linkers set forth in Tables 3 and 4. In another embodiment the linker is selected from phosphate and sulfate linkers.

Examples of such modified neuroactive steroids include those having the structures set forth in Formulas V-VII, wherein R is selected from a neuroactive steroid, a therapeutic agent, a modifying moiety, and combinations thereof; L is a sulfur- or phosphorus-containing linker; n is an integer equal to, or greater than, 1; R₂ is ═O or —OH; R₃ is —H or —OH; R₄ is —H or —OH, and is an optional C═C bond. Preferably, n is an integer from 1 to 50.

Some non-limiting examples of modified neuroactive steroids using phosphorus- or sulfur-containing linkers and using pregnenolone as the neuroactive steroid have the following structures. It should be understood that similar modifications can be made to the other neuroactive steroids described herein, and that such modified neuroactive steroids are also within the scope of the present disclosure.

wherein R is selected from a neuroactive steroid, a therapeutic agent, and a modifying moiety. In a preferred embodiment, the phosphorus- or sulfur-containing linkers form a phosphate or sulfate linkage with the R moiety.

As discussed herein, in some embodiments, a neuroactive steroid may be linked to another neuroactive steroid. Some non-limiting illustrative examples of modified neuroactive steroids comprising a neuroactive steroid linked to another neuroactive steroid via a linker, are shown below, using pregnenolone as the neuroactive steroid. It should be understood that similar modifications can be made to the other neuroactive steroids described herein, and that such modified neuroactive steroids are also within the scope of the present disclosure. It should further be understood that although only one regioisomer is shown in the following examples, this should not be construed as limiting.

In these embodiments, w is 0-20; m is 2-20; and R′ is H, a modifying moiety, a neuroactive steroid (e.g., another pregnenolone or other neuroactive steroid), a therapeutic agent, or a simple (e.g., lower) carboxyl alkyl. Optionally, the linkers may be modified by placing nitrogen substituents along the chain in the linkers.

In one particular embodiment, the modified neuroactive steroid has the following structure when pregnenolone is used as an exemplary neurosteroid:

wherein D=—H, —CH₃, —CO₂H, —CO₂R′, —CH₂CO₂H, —CH₂CO₂R′, and R′ is H, a therapeutic agent, a modifying moiety, a neuroactive steroid, or an alkyl.

Some additional non-limiting illustrative examples of modified neuroactive steroids using pregnenolone as the neuroactive steroid are shown below:

wherein R is a prodrug or a poly-pharma; Q is C, O, S, or N; R₁, R₃, and R₄ are independently selected from C, ═O, —OH, —NH₂, —OR, —NR, and R; and n is an integer of 0 to 50. As used in this context, the term “poly-pharma” indicates that the R substituent may be a neuroactive steroid, therapeutic agent, or modifying moiety having the same or different pharmacologic activity as the pregnenolone (or other neuroactive steroid) to which it is linked.

As discussed herein, in some embodiments, the modified neuroactive steroid is linked to a pharmacologically active group (e.g., the R moiety is a therapeutic agent). Suitable pharmacologically active groups include, but are not limited to, various medications and therapeutic agents. In such embodiments, the modified neuroactive steroid comprises the general formula R-Lₙ-NS, wherein R=a therapeutic agent, n=an integer of 0 to 50, and NS=a neuroactive steroid.

In embodiments where n═O, the therapeutic agent is directly linked to the neuroactive steroid (i.e., there is no linker). In this embodiment, the therapeutic agent will typically comprise a carboxylic acid and will be attached to the neuroactive steroid through an ester linkage. Examples of this type of attachment are described above in the section on direct attachment. In one embodiment, the therapeutic agent is directly attached to the neuroactive steroid at the C3 position of the neuroactive steroid.

In other embodiments, n=an integer of 1 to 50, and the therapeutic agent is attached to the neuroactive steroid via a linker. In preferred embodiments, R=a therapeutic agent and NS=pregnenolone as shown below:

In other embodiments, the therapeutic agent is attached to the neuroactive steroid at the C20 position. An example of such a structure where R=a therapeutic agent and NS=pregnenolone is shown below. In this embodiment, the C3 position (represented by R′) may be —OH, or optionally may be substituted with an ester, such as a C₃-C₂₄ carboxylic acid ester, to provide stability to the structure:

Other neuroactive steroids can be modified in a similar manner.

As used herein, the term “therapeutic agent” refers to a drug or medicine. Suitable therapeutic agents include, but are not limited to, antipsychotics, such as clozapine (e.g., Clozaril), aripiprazole (e.g., Abilify) olanzapine (e.g., Zyprexa), quetiapine (e.g., Seroquel), perphenazine (e.g., Trilafon), ziprasidone (e.g., Geodon), risperidone (e.g., Risperidal), haloperidol (e.g., Haldol), fluphenazine (e.g.,Prolixin), lurasidone (e.g., Latuda), paliperidone (e.g., Invega), and asenapine (e.g., Saphris); antidepressants, such as fluoxetine (e.g., Prozac), sertraline (e.g., Zoloft), paroxetine (e.g., Paxil/Paxil CR), buproprion (e.g., Wellbutrin/Zyban), citalopram (e.g., Celexa/Escitalopram [Lexopro]), venlafaxine (e.g., Effexor), venlafaxine extended release (e.g., Effexor XR), fluvoxamine (e.g., Luvox), duloxetine (e.g., Cymbalta), mirtazapine (e.g., Remeron), trazodone (e.g., Desyrel), desvenlaxfaxine succinate (e.g., Prestiq); mood stabilizers, such as divalproex sodium, and extended release (e.g., Depakote/Depakote ER), valproic acid (e.g., Depakene), lamotrigine (e.g., Lamictal), topiramate (e.g., Topamax), carbamazepine (e.g., Tegretol), oxcarbazepine (e.g., Trileptil), tiagabine (Gabatril), gabapentin (e.g., Neurontin); those used to treat substance use disorders, such as naltrexone (e.g., ReVia or Vivitrol), buproprion (e.g., Zyban), nicotine replacement (e.g., patch, inhaler, varenicline (e.g., Chantix), etc.), alcohol dependence (e.g., acamprosate (e.g., Campral), baclofen (e.g., Lioresal), etc.); those used to treat Alzheimer's Disease, such as donepezil (e.g., Aricept), galantamine (e.g., Reminyl), rivastigmine (e.g., Exelon), mematine (e.g., Namenda); those used to treat ADHD, such as methylphenidate (e.g., Ritalin), dextroamphetamine (e.g., Dexedrine), dextroamphetamine/amphetamine (e.g., Adderall), guanfacine/guanfacine extended release (e.g., Tenex/Intuniv), atomoxetine (e.g., Strattera); anxiolytics, such as hydroxyzine (e.g., Vistaril, Atarax), buspirone (e.g., Buspar); pain disorders and/or anti-inflammatory actions, such as acetaminophen (e.g., Tylenol), ibuprofen (e.g., Motrin), other NSAIDs (non-steroidal anti-inflammatory drugs), aspirin, naproxen (e.g., Naprosyn, Aleve), indomethacin (e.g., Indocin), buprenorphine (e.g., Suboxone, Naloxone), prednisone, prednisolone; lipid lowering drugs, such as statins (e.g., simvastatin (e.g., Zocor), atorvastatin (e.g., Lipitor), lovastatin (e.g., Mevacor), pravastatin (e.g., Pravachol), niacin; antihypertensives, such as propranol, ACE inhibitors (e.g., lisinopril (e.g., Prinivil, Zestril), calcium channel blockers, i.e., nifedipine (e.g., Adalat, Procardia), diuretics; as well as medications used to treat other conditions, such as hematology applications, other neurodegenerative disorders, such as multiple sclerosis, Niemann-Pick Type C, stroke, ocular conditions, such as glaucoma and macular degeneration, and cancer (e.g., Gleevec).

Examples of some suitable modified neuroactive steroids conjugated with a medication include the following. Although the following examples use pregnenolone as the neuroactive steroid, similar modifications made to other neuroactive steroids are also within the scope of the present disclosure. Further, similar neuroactive steroid-medication conjugations made using any of the phosphorus- or sulfur-containing linkers described herein are also within the scope of the present disclosure.

A. Pregnenolone Conjugated with Clozapine/Olanzapine

B. Pregnenolone Conjugated with Aripiprazole

C. Pregnenolone Conjugated with Fluoxetine

D. Pregnenolone Conjugated with Sertraline

In other embodiments, R=a modifying moiety. Modifying moieties are moieties that modify the modified neuroactive steroid, and provide the compound with desired properties as described herein. For example, the modifying moiety can reduce the rate of degradation of the modified neuroactive steroid in various environments (such as in tissues such as skin, the GI tract, and/or the bloodstream), such that less of the modified neuroactive steroid is degraded in the modified form than would be degraded in the absence of the modifying moiety in such environments. Alternatively, the modifying moiety can be used to control the rate of release to optimize sustained release of the neuroactive steroid. Preferred modifying moieties are those which permit the modified neuroactive steroid to retain a therapeutically significant percentage of the biological activity of the native neuroactive steroid.

Exemplary modifying moieties that may be attached to the neuroactive via a linker include any of those described previously as suitable for direct attachment to the neuroactive steroid. For instance, the modifying moiety may be designed to target specific cells/tissues, such as the chemical delivery system group described herein. The modifying moiety may also include moieties that affect the extended or sustained release properties of the neuroactive steroid, such as the esters described above. The modifying moieties may be prodrugs or poly-pharmas.

Other examples of suitable modifying moieties that may be linked to the neuroactive steroid are selected from the group consisting of hydrophilic moieties, polyalkylene glycolmoieties, sugar moieties, polysorbate moieties, biocompatible water-soluble moieties, polycationic moieties, bioadhesive polyanionic moieties, lipophilic moieties, amphophilic moieties, PEG/alkyl modifying moieties, salt forming moieties, and combinations thereof.

In some embodiments, the modifying moieties described below may be directly attached to the neuroactive steroid (i.e., there is no linker). In these embodiments, the modifying moiety will typically comprise a carboxylic acid and will be attached to the neuroactive steroid through an ester, carbonate, or a glycosidic bond. Examples of these types of attachment are described above in the section on direct attachment. In one embodiment, the modifying moiety is directly attached to the neuroactive steroid at the C3 position of the neuroactive steroid.

Moieties that Affect Stability, Solubility, and/or Biological Activity

There are numerous moieties that can be attached to the neuroactive steroid to form the modified neuroactive steroids described herein that modify the stability, solubility, and/or biological activity of the native neuroactive steroid. Examples include hydrophilic polymers or oligomers, amphiphilic polymers or oligomers, and lipophilic polymers or oligomers.

The polymers (or shorter chain oligomers) can include weak or degradable linkages in their backbones. For example, the polyalkylene glycols can include hydrolytically unstable linkages, such as lactide, glycolide, carbonate, ester, carbamate and the like, which are susceptible to hydrolysis. This allows the polymers to be cleaved into lower molecular weight fragments. Examples of such polymers are described, for example, in U.S. Pat. No. 6,153,211 to Hubbell et al.

Representative hydrophilic, amphiphilic, and lipophilic polymers and oligomers are described in more detail below.

Hydrophilic Moieties

The hydrophilic moiety may be various hydrophilic moieties as will be understood by those skilled in the art including, but not limited to, polyalkylene glycol moieties, other hydrophilic polymers, sugar moieties, polysorbate moieties, and combinations thereof.

Polyalkylene Glycol Moieties

Polyalkylene glycols are compounds with repeat alkylene glycol units. In some embodiments, the units are all identical (e.g., polyethylene glycol or polypropylene glycol). In other embodiments, the alkylene units are different (e.g., polyethylene-co-propylene glycol, or PLURONICS®). The polymers can be random copolymers (for example, where ethylene oxide and propylene oxide are co-polymerized) or branched or graft co-polymers.

Polyethylene glycol, or PEG, is a preferred polyalkylene glycol, and is useful in biological applications because it has highly desirable properties and is generally regarded as safe (GRAS) by the Food and Drug Administration. PEG has the formula—(CH₂CH₂O)ₙ—, where n can range from about 2 to about 4000 or more. PEG typically is colorless, odorless, water-soluble or water-miscible (depending on molecular weight), heat stable, chemically inert, hydrolytically stable, and generally nontoxic. PEG is also biocompatible, and typically does not produce an immune response in the body. Preferred PEG moieties of the invention include a number of PEG subunits selected from the following ranges shown in order of increasing preference: 2-50, 2-40, 2-30, 2-25, 2-20, 2-15, 2-10. In certain embodiments, the modifying moieties will include 2, 3, 4, 5, 6, 7, 8, 9, or 10 subunits.

The PEG may be monodispersed or polydispersed as commonly supplied on the market. By mono-dispersed, it is meant that the polyalkylene glycol can have a single molecular weight, or a relatively narrow range of molecular weights. One advantage of using the relatively low molecular weight, monodispersed polymers is that they form easily defined conjugate molecules, which can facilitate both reproducible synthesis and FDA approval.

The PEG can be a linear polymer with a hydroxyl group at each terminus (before being conjugated to the remainder of the neuroactive steroid). The PEG can also be an alkoxy PEG, such as methoxy-PEG (or mPEG), where one terminus is a relatively inert alkoxy group, while the other terminus is a hydroxyl group (that is coupled to the neuroactive steroid). The PEG can also be branched, which can in one embodiment be represented as R(—PEG-OH)ₘ in which R represents a central (typically polyhydric) core agent such as pentaerythritol or glycerol, and m represents the number of arms. Each branch can be different and can be terminated, for example, with ethers and/or esters. The number of arms m can range from three to a hundred or more, and one or more of the terminal hydroxyl groups can be coupled to the remainder of the neuroactive steroid, or otherwise subject to chemical modification. Other branched PEG include those represented by the formula (CH₃O-PEG-)_{pR-Z}, where p equals 2 or 3, R represents a central core such as Lys or glycerol, and Z represents a group such as carboxyl that is subject to ready chemical activation. Still another branched form, the pendant PEG, has reactive groups, such as carboxyls, along the PEG backbone rather than, or in addition to, the end of the PEG chains. Forked PEG can be represented by the formula PEG(-LCHX₂)ₙ is another form of branched PEG, where L is a linking group and X is an activated terminal group. The term polyethylene glycol or PEG represents or includes all forms of linear or branched PEG, and polyalkalene glycol or PEG includes all forms of linear or branched PEG.

Sugar Moieties

The modified neuroactive steroids described herein can include sugar moieties, as such as known by those skilled in the art. In general, the sugar moiety is a carbohydrate product of at least one saccharose group. Representative sugar moieties include, but are not limited to, glycerol moieties, mono-, di-, tri-, and oligosaccharides, and polysaccharides such as starches, glycogen, cellulose and polysaccharide gums. Specific monosaccharides include C₆ and above (preferably C₆ to CO sugars such as glucose, fructose, mannose, galactose, ribose, and sedoheptulose; di- and trisaccharides include moieties having two or three monosaccharide units (preferably C₅ to C₈ such as sucrose, cellobiose, maltose, lactose, and raffinose. Sugar moieties may be attached to a neuroactive steroid either through use of a linker, or directly (i.e., no linker) using the techniques described herein. Conjugation using sugar moieties is described in U.S. Pat. Nos. 5,681,811, 5,438,040, and 5,359,030, the entire disclosures of which are incorporated herein by reference.

Polysorbate Moieties

The polysorbate moiety may be various polysorbate moieties as will be understood by those skilled in the art including, but are not limited to, sorbitan esters, and polysorbate derivatized with polyoxyethylene. Conjugation using polysorbate moieties is described in U.S. Pat. Nos. 5,681,811, 5,438,040, and 5,359,030, the entire disclosures of which are incorporated herein by reference.

Biocompatible Water-Soluble Polycationic Moieties

In some embodiments, biocompatible water-soluble polycationic polymers can be used. Biocompatible water-soluble polycationic polymers include, for example, any polymer having protonated heterocycles attached as pendant groups. “Water soluble” means that the entire polymer is soluble in aqueous solutions, such as buffered saline or buffered saline with small amounts of added organic solvents as cosolvents, at a temperature between 20 and 37° C. In some embodiments, the polymer itself is not sufficiently soluble in aqueous solutions per se but is brought into solution by grafting with water-soluble polymers such as PEG chains. Examples include polyamines having amine groups on either the polymer backbone or the polymer sidechains, such as poly-L-Lys and other positively charged polyamino acids of natural or synthetic amino acids or mixtures of amino acids, including poly(D-Lys), poly(ornithine), poly(Arg), and poly(histidine), and nonpeptide polyamines such as poly(aminostyrene), poly(aminoacrylate), poly(N-methyl aminoacrylate), poly(N-ethylaminoacrylate), poly(N,N-dimethyl aminoacrylate), poly(N,N-diethylaminoacrylate), poly(aminomethacrylate), poly(N-methyl amino-methacrylate), poly(N-ethyl aminomethacrylate), poly(N,N-dimethyl aminomethacrylate), poly(N,N-diethyl aminomethacrylate), poly(ethyleneimine), polymers of quaternary amines, such as poly(N,N,N-trimethylaminoacrylate chloride), poly(methyacrylamidopropyltrimethyl ammonium chloride), and natural or synthetic polysaccharides such as chitosan. In preferred embodiments, the biocompatible Water-Soluble polycationic moiety is the amino acid serine or glycine.

Other Hydrophilic Moieties

Other hydrophilic polymers can also be used. Examples include poly(oxyethylated polyols) such as poly(oxyethylated glycerol), poly(oxyethylated sorbitol), and poly(oxyethylated glucose); poly(vinyl alcohol) (“PVA”); dextran; carbohydrate-based polymers and the like. The polymers can be homopolymers or random or block copolymers and terpolymers based on the monomers of the above polymers, linear chain or branched.

Specific examples of suitable additional polymers include, but are not limited to, poly(oxazoline), difunctional poly(acryloylmorpholine) (“PAcM”), and poly(vinylpyrrolidone) (“PVP”). PVP and poly(oxazoline) are well known polymers in the art and their preparation should be readily apparent to the skilled artisan. PAcM and its synthesis and use are described in U.S. Pat. Nos. 5,629,384 and 5,631,322, the disclosures of which are incorporated herein by reference in their entirety.

Bioadhesive Polyanionic Moieties

Certain hydrophilic polymers appear to have potentially useful bioadhesive properties. Examples of such polymers are found, for example, in U.S. Pat. No. 6,197,346 to Mathiowitz, et al. Those polymers containing carboxylic groups (e.g., poly(acrylic acid)) exhibit bioadhesive properties, and also are readily conjugated with the neuroactive steroid described herein. Rapidly bioerodible polymers that expose carboxylic acid groups on degradation, such as poly(lactide-co-glycolide), polyanhydrides, and polyorthoesters, are also bioadhesive polymers. These polymers can be used to deliver the modified neuroactive steroids to the gastrointestinal tract. As the polymers degrade, they can expose carboxylic acid groups to enable them to adhere strongly to the gastrointestinal tract, and can aid in the delivery of the modified neuroactive steroids.

Lipophilic Moieties

In some embodiments, the modifying moiety comprises a lipophilic moiety. The lipophilic moiety may be various lipophilic moieties as will be understood by those skilled in the art including, but not limited to, alkyl moieties, alkenyl moieties, alkynyl moieties, aryl moieties, arylalkyl moieties, alkylaryl moieties, fatty acid moieties, adamantantyl, and cholesteryl, as well as lipophilic polymers and/or oligomers.

The alkyl moiety can be a saturated or unsaturated, linear, branched, or cyclic hydrocarbon chain. In some embodiments, the alkyl moiety has at least 1, 2, 3, or more carbon atoms. In other embodiments, the alkyl moiety is a linear, saturated or unsaturated alkyl moiety having between 1, 2, 3, 4, 5, 6, 7, 8, 9, 10, 11, 12, 13, 14, 15, 16, 17, 18, 19 or 20 carbon atoms. Examples include saturated, linear alkyl moieties such as methyl, ethyl, propyl, butyl, pentyl, hexyl, heptyl, octyl, nonyl, decyl, undecyl, dodecyl, tridecyl, tetradecyl, pentadecyl, hexadecyl, octadecyl, nonadecyl and eicosyl; saturated, branched alkyl moieties such as isopropyl, sec-butyl, tert-butyl, 2-methylbutyl, tert-pentyl, 2-methyl-pentyl, 3-methylpentyl, 2-ethylhexyl, 2-propylpentyl; and unsaturated alkyl moieties derived from the above saturated alkyl moieties including, but not limited to, vinyl, allyl, 1-butenyl, 2-butenyl, ethynyl, 1-propynyl, and 2-propynyl. In other embodiments, the alkyl moiety is a lower alkyl moiety. In still other embodiments, the alkyl moiety is a C₁ to C₃ lower alkyl moiety. In some embodiments, the modifying moiety specifically does not consist of an alkyl moiety, or specifically does not consist of a lower alkyl moiety, or specifically does not consist of an alkane moiety, or specifically does not consist of a lower alkane moiety.

The alkyl groups can either be unsubstituted or substituted with one or more substituents, and such substituents preferably either do not interfere with the methods of synthesis of the conjugates or eliminate the biological activity of the conjugates. Potentially interfering functionality can be suitably blocked with a protecting group so as to render the functionality non-interfering. Each substituent may be optionally substituted with additional non-interfering substituents. The term “non-interfering” characterizes the substituents as not adversely affecting any reactions to be performed in accordance with the process of this invention.

The fatty acid moiety may be various fatty acid moieties including natural or synthetic, saturated or unsaturated, linear or branched fatty acid moieties. In some embodiments, the fatty acid moiety has at least 2, 3, 4, or more carbon atoms. In other embodiments, the fatty acid moiety has 2, 3, 4, 5, 6, 7, 8, 9, 10, 11, 12, 13, 14, 15, 16, 17, 18, 19, 20, 21, 22, 23, or 24 carbon atoms.

When the modifying moiety is an aryl ring, the ring can be functionalized with a nucleophilic functional group (such as OH, SH, or NHR′) that is positioned so that it can react in an intramolecular fashion with the carbamate moiety and assist in its hydrolysis. In some embodiments, the nucleophilic group is protected with a protecting group capable of being hydrolyzed or otherwise degraded in vivo, with the result being that when the protecting group is deprotected, hydrolysis of the conjugate, and resultant release of the parent neuroactive steroid, is facilitated.

Amphiphilic Moieties

In some embodiments, the modifying moiety includes an amphiphilic moiety. Many polymers and oligomers are amphiphilic. These are often block co-polymers, branched copolymers or graft co-polymers that include hydrophilic and lipophilic moieties, which can be in the form of oligomers and/or polymers, such as linear chain, branched, or graft polymers or co-polymers.

The hydrophilic polymers or oligomers described may include combinations of any of the lipophilic and hydrophilic moieties described herein. Such polymers or oligomers typically include at least one reactive functional group, for example, halo, hydroxyl, amine, thiol, sulfonic acid, carboxylic acid, isocyanate, epoxy, ester, and the like, which are often at the terminal end of the polymer. These reactive functional groups can be used to attach a lipophilic linear or branched chain alkyl, alkenyl, alkynyl, arylalkyl, or alkylaryl group, or a lipophilic polymer or oligomer, thereby increasing the lipophilicity of the hydrophilic polymers or oligomers (and thereby rendering them generally amphiphilic).

The lipophilic groups can, for example, be derived from mono- or di-carboxylic acids, or where appropriate, reactive equivalents of carboxylic acids such as anhydrides or acid chlorides. Examples of suitable precursors for the lipophilic groups are acetic acid, propionic acid, butyric acid, valeric acid, isobutyric acid, trimethylacetic acid, caproic acid, caprylic acid, heptanoic acid, capric acid, pelargonic acid, lauric acid, myristic acid, palmitic acid, stearic acid, behenic acid, lignoceric acid, ceratic acid, montanoic acid, isostearic acid, isononanoic acid, 2-ethylhexanoic acid, oleic acid, ricinoleic acid, linoleic acid, linolenic acid, erucic acid, soybean fatty acid, linseed fatty acid, dehydrated castor fatty acid, tall oil fatty acid, tung oil fatty acid, sunflower fatty acid, safflower fatty acid, acrylic acid, methacrylic acid, maleic anhydride, orthophthalic anhydride, terephthalic acid, isophthalic acid, adipic acid, azelaic acid, sebacic acid, tetrahydrophthalic anhydride, hexahydrophthalic anhydride, succinic acid and polyolefin carboxylic acids.

The terminal lipophilic groups need not be equivalent, i.e., the resulting copolymers can include terminal lipophilic groups that are the same or different. The lipophilic groups can be derived from more than one mono or di-functional alkyl, alkenyl, alkynyl, cycloalkyl, arylalkyl or alkylaryl group as defined above.

PEG/Alkyl Modifying Moieties

The modifying moiety may be a linear or branched polymeric moiety comprising one or more linear or branched polyalkylene glycol moieties and/or one or more linear or branched, substituted or unsubstituted alkyl moieties. However, in certain embodiments, the modifying moiety specifically does not consist of an alkyl moiety and in other embodiments, the modifying moiety specifically does not consist of an alkane moiety. The polyalkylene glycol moieties in some embodiments include 1, 2, 3, 4, 5, 6, 7, 8, 9, 10, 11, 12, 13, 14, 15, 16, 17, 18, 19, 20, 21, 22, 23, 24, or 25 PAG subunits, preferably PEG or PPG subunits or combinations thereof. The alkyl moieties are saturated or unsaturated and are preferably 1, 2, 3, 4, 5, 6, 7, 8, 9, 10, 11, 12, 13, 14, 15, 16, 17, 18, 19, or 20 carbon atoms. The alkyl moieties are preferably alkane moieties.

The pharmaceutical characteristics, such as hydrophilicity/lipophilicity of the modified neuroactive steroid can be varied by adjusting the number of PEG monomers, the type and length of alkyl chain, the nature of the PEG-peptide linkage, and the number of conjugation sites. The exact nature of the PEG-peptide linkage can be varied such that it is stable and/or sensitive to hydrolysis at physiological pH or in plasma.

Salt-Forming Moieties

In some embodiments, the modifying moiety comprises a salt-forming moiety. The salt-forming moiety may be various suitable salt-forming moieties as will be understood by those skilled in the art including, but not limited to, carboxylate and ammonium. In some embodiments wherein the modifying moiety includes a salt forming moiety, the modified neuroactive steroid is provided in salt form. In these embodiments, the modified neuroactive steroid is associated with a suitable pharmaceutically acceptable counterion as will be understood by those skilled in the art including, but not limited to, negative ions such as chloro, bromo, iodo, phosphate, acetate, carbonate, sulfate, tosylate, and mesylate, or positive ions such as sodium, potassium, calcium, lithium, and ammonium.

The modifying moiety can include any hydrophilic moieties, lipophilic moieties, amphiphilic moieties, salt-forming moieties, and combinations thereof. In preferred embodiments, the modifying moiety is selected from the group consisting of (CH₂CH₂O)ₚCH₃ where p is 0, 1, 2, 3, 4, 5, 6, 7, 8, 9, 10, 11, 12, 13, 14, 15, 16, 17, 18, 19 or 20; (CH₂)_{q}CH₃ where q is 0, 1, 2, 3, 4, 5, 6, 7, 8, 9, 10, 11, 12, 13, 14, 15, 16, 17, 18, 19 or 20; CH₂CH₂(OCH₂CH₂)ᵣOH where r is 0, 1, 2, 3, 4, 5, 6, 7, 8, 9, 10, 11, 12, 13, 14, 15, 16, 17, 18, 19 or 20; C(CH₂OH)₃; CH(CH₂OH)₂; C(CH₃)₃; CH(CH₃)₂; CH₂CH₂(OCH₂CH₂)ₛC(O)(CH₂)ₜCH₃ where s is 0, 1, 2, 3, 4, 5, 6, 7, 8, 9, 10, 11, 12, 13, 14, 15, 16, 17, 18, 19 or 20 and t is 0, 1, 2, 3, 4, 5, 6, 7, 8, 9, 10, 11, 12, 13, 14, 15, 16, 17, 18, 19 or 20; and (CH₂CH₂O)_{y}C(O)(CH₂)_{z}CH₃ where y is 0, 1, 2, 3, 4, 5, 6, 7, 8, 9, 10, 11, 12, 13, 14, 15, 16, 17, 18, 19 or 20 and z is 0, 1, 2, 3, 4, 5, 6, 7, 8, 9, 10, 11, 12, 13, 14, 15, 16, 17, 18, 19 or 20.

The foregoing examples of modifying moieties for specific purposes is intended as illustrative of the invention and should not be taken as limiting in any way. One skilled in the art will recognize that suitable moieties for conjugation to achieve particular functionality will be possible within the bounds of the chemical conjugation mechanisms disclosed and claimed herein. Accordingly, additional moieties can be selected and used according to the principles of the invention as disclosed herein.

III. Compounds Based on Derivatives in the Neuroactive Steroid Core Ring Skeleton And Substitution Therein

Another aspect of the present disclosure provides a modified neuroactive steroid which comprises a neuroactive steroid whose core has been modified to add metabolic stabilizing. The addition of such modifications will decrease the metabolism of the neuroactive steroid and thus increase its duration of action. Some examples of such modified neuroactive steroids include, but are not limited to, modified neuroactive steroids having any of the following structures (Formulas VIII-X):

wherein:

R₁ is F, CH₃, or H,

R₂ is F, CH₃, or H,
denotes an optional double bond,

X is N or C,

Y is N or C,

Z is C, O, S, or N, and

W is C, O, S, or N.

In embodiments where the modified neuroactive steroid has a structure as set forth in Formula VIII, R₁ and R₂ are not both H, and R₂ is not H when R₁ is F at the C17 position and is a C═C bond (i.e., when there is a C═C bond between C5-C6). In embodiments where the modified neuroactive steroid has a structure as set forth in Formula IX, W, X, Y, and Z are not all C. In embodiments where the modified neuroactive steroid has a structure as set forth in Formula X, if X, Y, and Z are all C, then R₁ and R₂ are not both H, and if R₁ and R₂ are both H, then X, Y, and Z are not all C.

Exemplary steroids that may be modified in this manner include, but are not limited to, any of the following: pregnenolone, allopregnanolone, epiallopregnanolone, epipregnanolone, and the like.

In one embodiment, R₂ is H, and R₁ is F and is attached at the C17 position of the neuroactive steroid. Some exemplary modified neuroactive steroids having this structure are as follows:

The Formula XI modified neuroactive steroid may be prepared using the techniques set forth in Jensen, Steroids (1976), Vol. 28(4), p. 437-447, which is herein incorporated by reference. These techniques may also be used to modify allopregnanolone to prepare the modified neuroactive steroid having Formula XII.

Due to the central nature of many neuroactive steroids in steroid biosynthesis (e.g., pregnenolone), care must be taken not to perturb the neurosteroid structure such that a negative outcome of steroid biochemistry and steroid biosynthesis may result. Those skilled in the art have demonstrated some ways to modify the core structure without disrupting steroid biosynthesis, those methods of which are hereby incorporated by reference (see.e. g., Duran, F. J., et al. (2006) Tetrahedron 62:4762-4768).

### Pharmaceutical Compositions and Formulations

As used herein, the term “pharmaceutical composition” means physically discrete coherent portions suitable for medical administration. The term “dosage unit form” or “unit dosage” means physically discrete coherent units suitable for medical administration, each containing a daily dose or a multiple (up to four times) or a sub-multiple (down to a fortieth) of a daily dose of the active compound in association with a carrier and/or enclosed within an envelope. Whether the composition contains a daily dose, or for example, a half, a third or a quarter of a daily dose, will depend on whether the pharmaceutical composition is to be administered once or, for example, twice, three times or four times a day, respectively.

The modified neuroactive steroids of the present disclosure may be administered to the subject as a composition which comprises a pharmaceutically effective amount of the modified neuroactive steroids and an acceptable carrier and/or excipients. The composition may comprise one modified neuroactive steroid, or alternately, may comprise a combination of two or more different neuroactive steroids. For example, the composition may comprise two or more different types of modified pregnenolone (e.g., pregnenolone heptanoate and pregnenolone decanoate). In another example, the composition may comprise a modified pregnenolone and a modified allopregnenolone. Other suitable combinations of the modified neuroactive steroids described herein may also be used.

A pharmaceutically acceptable carrier includes any solvents, dispersion media, or coatings that are physiologically compatible. Preferably, the carrier is suitable for intravenous, intramuscular, oral, intraperitoneal, intradermal, transdermal, topical, nasal or subcutaneous administration. One exemplary pharmaceutically acceptable carrier is physiological saline. Other pharmaceutically acceptable carriers and their formulations are well-known and generally described in, for example, Remington's Pharmaceutical Science (18th Ed., ed. Gennaro, Mack Publishing Co., Easton, Pa., 1990). Various pharmaceutically acceptable excipients are well-known in the art and can be found in, for example, Handbook of Pharmaceutical Excipients (4th ed., Ed. Rowe et al. Pharmaceutical Press, Washington, D.C.). The composition can be formulated as a solution, microemulsion, liposome, capsule, tablet, or other suitable forms. The active component which comprises the modified neuroactive steroid may be coated in a material to protect it from inactivation by the environment prior to reaching the target site of action. The pharmaceutical compositions of the present disclosure are preferably sterile and non-pyrogenic at the time of delivery, and are preferably stable under the conditions of manufacture and storage.

In other embodiments of the present disclosure, the pharmaceutical compositions are regulated-release formulations. Modified neuroactive steroids of the present disclosure may be admixed with biologically compatible polymers or matrices which control the release rate of the copolymers into the immediate environment. Controlled or sustained release compositions include formulation in lipophilic depots (e.g., fatty acids, waxes, oils).

In some embodiments of the present disclosure, pharmaceutical compositions comprise modified neuroactive steroids formulated with oil and emulsifier to form water-in-oil microparticles and/or emulsions. The oil may be any non-toxic hydrophobic material liquid at ambient temperature to about body temperature, such as edible vegetable oils including safflower oil, soybean oil, corn oil, and canola oil; or mineral oil. Chemically defined oil substance such as lauryl glycol may also be used. The emulsifier useful for this embodiment includes Span 20 (sorbitan monolaurate) and phosphatidylcholine. In some embodiments, a modified neuroactive steroid composition is prepared as an aqueous solution and is prepared into an water-in-oil emulsion dispersed in 95 to 65% oil such as mineral oil, and 5 to 35% emulsifier such as Span 20. In another embodiment of the disclosure, the emulsion is formed with alum rather than with oil and emulsifier. These emulsions and microparticles reduce the speed of uptake of modified neuroactive steroids, and achieve controlled delivery. In other embodiments, the pharmaceutical compositions also include additional therapeutically active agents (i.e., medications) as described herein.

The present disclosure further provides a kit comprising (i) a composition comprising a modified neuroactive steroid and (ii) instructions for administering the composition to a subject in need thereof at intervals greater than 24 hours, more preferably greater than 36 hours, for the treatment of disorders of the nervous system. In another preferred embodiment, the modified neuroactive steroid is formulated in dosages for administration of greater than about 24, 30, 36, 42, 48, 54, 60, 66, 72, 78, 84, 90, 96, 102, 108, 114, 120, 126, 132, 138, 144, 150, 156, 162, 168, 174, 180, 186, 192, 198, 204, 210, 216, 222, 228, 234, or 240 hours, or any intervening interval thereof. In another embodiment of the kits described herein, the instructions indicate that the modified neuroactive steroid is to be administered every about 24, 30, 36, 42, 48, 54, 60, 66, 72, 78, 84, 90, 96, 102, 108, 114, 120, 126, 132, 138, 144, 150, 156, 162, 168, 174, 180, 186, 192, 198, 204, 210, 216, 222, 228, 234, or 240 hours, or any interval in between. Kits may comprise additional components, such as packaging and one or more apparatuses for the administration of the modified neuroactive steroid, such as a hypodermic syringe.

In general, an embodiment of the present disclosure is to administer a suitable dose of a therapeutic modified neuroactive steroid composition that will be the lowest effective dose to produce a therapeutic effect, for example, mitigating symptoms. The therapeutic modified neuroactive steroid compositions are preferably administered at a dose per subject, which corresponds to a dose per day of at least about 2 mg, at least about 5 mg, at least about 10 mg, or at least about 20 mg as appropriate minimal starting dosages, or about x mg, wherein x is an integer between 1 and 20. In one embodiment of the methods described herein, a dose of about 0.01 to about 500 mg/kg can be administered. In general, the effective dosage of the compound of the present disclosure is about 50 to about 1000 micrograms of the compound per kilogram of the subject per day. In one specific embodiment, the equivalent dosage per day, regardless of the frequency with which the doses are administered, is from about 5 to 500, or more preferably, from about 10 to 400 mg/day.

However, it is understood by one skilled in the art that the dose of the composition of the present disclosure will vary depending on the subject and upon the particular route of administration used. It is routine in the art to adjust the dosage to suit the individual subjects. Additionally, the effective amount may be based upon, among other things, the size of the compound, the biodegradability of the compound, the bioactivity of the compound and the bioavailability of the compound. If the compound does not degrade quickly, is bioavailable and highly active, a smaller amount will be required to be effective. The actual dosage suitable for a subject can easily be determined as a routine practice by one skilled in the art, for example a physician or a veterinarian given a general starting point. For example, the physician or veterinarian could start doses of the compound of the invention employed in the pharmaceutical composition at a level lower than that required in order to achieve the desired therapeutic effect, and increase the dosage with time until the desired effect is achieved.

In the context of the present disclosure, the term “treatment regimen” is meant to encompass therapeutic, palliative and prophylactic modalities of administration of one or more compositions comprising one or more modified neuroactive steroid. A particular treatment regimen may last for a period of time which will vary depending upon the nature of the particular disease or disorder, its severity and the overall condition of the patient, and may extend from once daily, or more preferably once every 36 hours or 48 hours or longer, to once every month or several months. Following treatment, the patient is monitored for changes in his/her condition and for alleviation of the symptoms of the disorder or disease state. The dosage of the modified neuroactive steroids may either be increased in the event the patient does not respond significantly to current dosage levels, or the dose may be decreased if an alleviation of the symptoms of the disorder or disease state is observed, or if the disorder or disease state has been ablated, or if an unacceptable side effects are seen with the starting dosage.

In one embodiment, a therapeutically effective amount of the modified neuroactive steroid is administered to the subject in a treatment regimen comprising intervals of at least 36 hours, or more preferably 48 hours, between dosages. In another embodiment, the modified neuroactive steroid is administered at intervals of at least 54, 60, 66, 72, 78, 84, 90, 96, 102, 108, 114, 120, 126, 132, 138, 144, 150, 156, 162, 168, 174, 180, 186, 192, 198, 204, 210, 216, 222, 228, 234, or 240 hours, or the equivalent amount of days. In some embodiments, the agent is administered every other day, while in other embodiments it is administered weekly. If two modified neuroactive steroids are administered to the subject, such modified neuroactive steroids may be administered at the same time, such as simultaneously, or essentially at the same time, such as in succession. Alternatively, their administration may be staggered. For example, two modified neuroactive steroids which are each administered every 48 hours may both be administered on the same days, or one may be administered one day and the other on the next day and so on in an alternating fashion.

In other embodiments, the modified neuroactive steroid is administered in a treatment regimen which comprises at least one uneven time interval, wherein at least one of the time intervals is at least 24, 30, 36, 42, 48, 54, 60, 66, 72, 78, 84, 90, 96, 102, 108, 114, 120, 126, 132, 138, 144, 150, 156, 162, 168, 174, 180, 186, 192, 198, 204, 210, 216, 222, 228, 234, or 240 hours, or the equivalent amount of days.

In one embodiment, the modified neuroactive steroid is administered to be subject at least three times during a treatment regimen, such that there are at least two time intervals between administrations. These intervals may be denoted I1 and 12. If the modified neuroactive steroid is administered four times, then there would be an additional interval between the third and fourth administrations, 13, such that the number of intervals for a given number “n” of administrations is n−1. Accordingly, in one embodiment, at least one of the time intervals between administrations is greater than about 24, 30, 36, 42, 48, 54, 60, 66, 72, 78, 84, 90, 96, 102, 108, 114, 120, 126, 132, 138, 144, 150, 156, 162, 168, 174, 180, 186, 192, 198, 204, 210, 216, 222, 228, 234, or 240 hours. In another embodiment, at least 1%, 2%, 3%, 4%, 5%, 10%, 15%, 20%, 25%, 30%, 40%, 50%, 60%, 70%, 80%, 90% or 95% of the total number n−1 of time intervals are at least about 24, 30, 36, 42, 48, 54, 60, 66, 72, 78, 84, 90, 96, 102, 108, 114, 120, 126, 132, 138, 144, 150, 156, 162, 168, 174, 180, 186, 192, 198, 204, 210, 216, 222, 228, 234, or 240 hours.

In yet another embodiment, the average time interval between administrations ((I₁+I₂+ . . . +Iₙ₋₁)/n−1) is at least 24, 30, 36, 42, 48, 54, 60, 66, 72, 78, 84, 90, 96, 102, 108, 114, 120, 126, 132, 138, 144, 150, 156, 162, 168, 174, 180, 186, 192, 198, 204, 210, 216, 222, 228, 234, or 240 hours, or at least two weeks.

In another embodiment, the dosage regimen consists of two or more different interval sets. For example, a first part of the dosage regimen is administered to a subject daily, every other day, or every third day, for example, at about 22 mg modified neuroactive steroid/m² body surface area of the subject, wherein the subject is a human. In some embodiment of the invention, the dosing regimen starts with dosing the subject every other day, every third day, weekly, biweekly, or monthly. The dosage for administration every other day or every third day may be up to about 65 mg/m² and 110 mg/m² respectively. For a dosing regimen comprising dosing of the modified neuroactive steroid every week, the dose comprises up to about 500 mg/m², and for a dosing regimen comprising dosing of the modified neuroactive steroid every two weeks or every month, up to 1.5 g/m² may be administered. The first part of the dosing regimen may be administered for up to 30 days, for example, 7, 14, 21, or 30 days. A subsequent second part of the dosing regimen with a different, longer interval administration with usually lower exposure (step-down dosage), administered weekly, every 14 days, or monthly may optionally follow, for example, at 500 mg/m² body surface area weekly, up to maximum of about 1.5 g/m² body surface area, continuing for 4 weeks up to two years, for example, 4, 6, 8, 12, 16, 26, 32, 40, 52, 63, 68, 78, or 104 weeks. Alternatively, if the disorder of the nervous system goes into remission or generally improves, the dosage may be maintained or kept at lower than maximum amount, for example, at 140 mg/m² body surface area weekly. If, during the step-down dosage regimen, the disease condition relapses, the first dosage regimen may be resumed until effect is seen, and the second dosing regimen may be implemented. This cycle may be repeated multiple times as necessary.

Any of the methods and means may be practiced using compositions and formulations described in this application.

In certain embodiment of the methods described herein, the route of administration can be oral, intraperitoneal, transdermal, subcutaneous, by intravenous or intramuscular injection, by inhalation, topical, intralesional, infusion; liposome-mediated delivery; topical, intrathecal, gingival pocket, rectal, intravaginal, intrabronchial, nasal, transmucosal, intestinal, ocular or otic delivery, or any other methods known in the art as one skilled in the art may easily perceive. Other embodiments of the compositions of the present disclosure incorporate particulate forms protective coatings, protease inhibitors or permeation enhancers for various routes of administration, including parenteral, pulmonary, nasal and oral. Administration can be systemic or local. In certain embodiments, the modified neuroactive steroid is administered orally.

For oral administration, the pharmaceutical preparation may be in liquid form, for example, solutions, syrups or suspensions, or may be presented as a drug product for reconstitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid). The pharmaceutical compositions may take the form of, for example, tablets or capsules prepared by conventional means with pharmaceutically acceptable excipients such as binding agents (e.g., pre-gelatinized maize starch, polyvinyl pyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well-known in the art.

When the modified neuroactive steroid is introduced orally, it may be mixed with other food forms and consumed in solid, semi-solid, suspension, or emulsion form; and it may be mixed with pharmaceutically acceptable carriers, including water, suspending agents, emulsifying agents, flavor enhancers, and the like. In one embodiment, the oral composition is enterically-coated. Use of enteric coatings is well known in the art. For example, Lehman (1971) teaches enteric coatings such as Eudragit S and Eudragit L. The Handbook of Pharmaceutical Excipients, 2^{nd} Ed., also teaches Eudragit S and Eudragit L applications. Preparations for oral administration may be suitably formulated to give controlled release of the active compound.

For buccal administration, the compositions may take the form of tablets or lozenges formulated in conventional manner. The compositions may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen free water, before use.

The compositions may also be formulated in compositions for administration via inhalation. For such administration, the compositions for use according to the present disclosure are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin, for use in an inhaler or insufflator may be formulated containing a powder mix of the compound and a suitable powder base such as lactose or starch.

In certain embodiments, compositions comprising modified neuroactive steroids are formulated in accordance with routine procedures as pharmaceutical compositions adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together. Where the composition is to be administered by infusion, it can be dispensed with an infusion bottle containing sterile pharmaceutical grade water or saline, with the intervals between administrations being greater than 24 hours, 32 hours, or more preferably greater than 36 or 48 hours. Where the composition is administered by injection, an ampoule of sterile water or saline for injection can be provided so that the ingredients may be mixed prior to administration.

In certain embodiments, the methods described herein allow continuous treatment of disorders of the nervous system by a sustained-release carrier such as transdermal patches, implantable medical devices coated with sustained-release formulations, or implantable or injectable pharmaceutical formulation suitable for sustained-release of the active components. In such embodiments, the intervals between administrations are preferably greater than 24 hours, 32 hours, or more preferably greater than 36 or 48 hours. For instance, an implantable device or a sustained released formulation which releases the modified neuroactive steroid over a 2 day period may the implanted every four days into the patient, such that the interval during which no modified neuroactive steroid is administered to the subject is 2 days. In related embodiments, the such interval where during which no administration occurs is at least 24+x hours, wherein x represents any positive integer.

In another embodiment, the modified neuroactive steroids are formulated to have a therapeutic effect when administered to a subject in need thereof at time intervals of at least 24 hours. In a specific embodiment, the modified neuroactive steroids are formulated for a long-lasting therapeutic affect such that a therapeutic effect in treating the disease is observed when the neuroactive steroids are administered to the subject at time intervals of at least 24, 30, 36, 42, 48, 54, 60, 66, 72, 78, 84, 90, 96, 102, 108, 114, 120, 126, 132, 138, 144, 150, 156, 162, 168, 174, 180, 186, 192, 198, 204, 210, 216, 222, 228, 234, or 240 hours between administrations.

In other embodiments of the methods described herein, additional therapeutically active agents are administered to the subject. In one embodiment, compositions comprising additional therapeutic agents(s) are administered to the subject as separate compositions from those comprising the modified neuroactive steroids. For example, a subject may be administered a composition comprising a modified neuroactive steroid subcutaneously while a composition comprising another therapeutic agent may be administered orally. The additional therapeutically active agents may treat the same disease as the modified neuroactive steroid, a related disease, or may be intended to treat an undesirable side effect of administration of the modified neuroactive steroid, such as to reduce swelling at a site of intradermal injection.

Methods of Administration and Treatment

The modified neuroactive steroids and pharmaceutical formulations exhibit one or more improved characteristics relative to the native neuroactive steroid and/or currently used medication, the modified neuroactive steroid can protect the biologically active nature of the neuroactive steroid component from degradation in various environments (such as the gastrointestinal tract (GI tract)), such that less of it is degraded in the unmodified form than would be degraded in the native form in such environments. In particular, certain modified forms of the disclosure can be orally administered in a dosage that ultimately provides a pharmaceutically acceptable amount of the biologically active neuroactive steroid in systemic circulation. That is to say, a sufficient amount of neuroactive steroid can survive in the GI tract and enter the bloodstream such that the biologically active neuroactive steroid is systemically present in a pharmacologically active amount sufficient to trigger a desired effect. Preferably, the addition of the modified neuroactive steroid improves the delivery of orally administered medications and/or neuroactive steroids into the bloodstream upon oral administration relative to the delivery of orally administered unmodified medications and/or neuroactive steroids into the bloodstream. More preferably, the improvement of the delivery of neuroactive steroid into the bloodstream for orally administered modified neuroactive steroid is at least 2 times the delivery of orally administered native neuroactive steroid, into the bloodstream. Still more preferably, the improvement of the delivery of modified neuroactive steroid into the bloodstream for orally administered modified neuroactive steroids is at least 3, 4, 5, 6, 7, 8, 9, 10, 15, 20, 25, 30, 40, 50, 60, 70, 80, 90, 100, 150, 200, 300, 400, or 500 times the delivery of orally administered unmodified medication and/or neuroactive steroid, into the bloodstream. Thus, administration of the modified neuroactive steroid of the disclosure can provide greater bioavailability of the biologically medicine and/or neuroactive steroid relative to administration of unmodified medicine and/or neuroactive steroid. Such delivery may increase patient compliance and/or reduce the needed amount of medicine and/or neuroactive steroid to achieve a therapeutic effect.

Thus, in one aspect, the disclosure provides a method of treating a phenotypic state of interest using a modified neuroactive steroid by administering to a subject in need thereof a therapeutically effective amount of a modified neuroactive steroid of the disclosure. The modified neuroactive steroid may be suitably administered by a variety of routes, including for example, parenteral and enteral routes. Examples of preferred routes include oral, subcutaneous, sublingual, buccal, nasal, intravenous and intramuscular.

Several approaches may be used in the use of the present modified neuroactive steroids for the treatment of a number of conditions. For example, it is envisioned that the modified neuroactive steroid can be presented as a monotherapy, preferably in an oral dosage form alone. Alternatively, the modified neuroactive steroids may be used together with more conventional therapeutic agents as part of a combination therapy. The primary categories of drugs that are currently used include the following: antipsychotics, such as clozapine (e.g., Clozaril), aripiprazole (e.g., Abilify) olanzapine (e.g., Zyprexa), quietiapine (e.g., Seroquel), perphenazine (e.g., Trilafon), ziprasidone (e.g., Geodon), risperidone (e.g., Risperidal), haloperidol (e.g., Haldol), fluphenazine (e.g., Prolixin), lurasidone (e.g., Latuda), paliperidone (e.g., Invega), and asenapine (e.g., Saphris); antidepressants, such as fluoxetine (e.g., Prozac), sertraline (e.g., Zoloft), paroxetine (e.g., Paxil/Paxil CR), buproprion (e.g., Wellbutrin/Zyban), citalopram (e.g., Celexa/Escitalopram [Lexopro]), venlafaxine (e.g., Effexor), venlaxfaxine extended release (e.g., Effexor XR), fluvoxamine (e.g., Luvox), duloxetine (e.g., Cymbalta), mirtazapine (e.g., Remeron), trazodone (e.g., Desyrel), and desvenlaxfaxine succinate (e.g., Prestiq); mood stabilizers, such as divalproex sodium, and extended release (e.g., Depakote/Depakote ER), valproic acid (e.g., Depakene), lamotrigine (e.g., Lamictal), topiramate (e.g., Topamax), carbamazepine (e.g., Tegretol), oxcarbazepine (e.g., Trileptil), tiagabine (Gabitril), gabapentin (e.g., Neurontin); those used to treat substance use disorders, such as naltrexone (e.g., ReVia or Vivitrol), buproprion (e.g., Zyban), nicotine replacement (e.g., patch, inhaler, varenicline (e.g., Chantix), etc.), alcohol dependence (e.g., acamprosate (e.g., Campral), baclofen (e.g., Lioresal), etc.); those used to treat Alzheimer's Disease, such as donepezil (e.g., Aricept), galatamine (e.g., Reminyl), rivastigmine (e.g., Exelon), mematine (e.g., Namenda); those used to treat ADHD, such as methylphenidate (e.g., Ritalin), dextroamphetiamine (e.g., Dexedrine), dextroamphetamine/amphetamine (e.g., Adderall), guanfacine/guanfacine extended release (e.g., Tenex/Intuniv), atomoxetine (e.g., Strattera); anxiolytics, such as hydroxyzine (e.g., Vistaril, Atarax), buspirone (e.g., Buspar); pain disorders and/or anti-inflammatory actions, such as acetaminophen (e.g., Tylenol), ibuprofen (e.g., motrin), other NSAIDs (non-steroidal anti-inflammatory drugs), aspirin, naproxen (e.g., Naprosyn, Aleve), indomethacin (e.g., Indocin), buprenorphine (e.g., Suboxone, Naloxone), prednisone, prednisolone; lipid lowering drugs, such as statins (e.g., simvastatin (e.g., Zocor), atorvastatin (e.g., Lipitor), lovastatin (e.g., Mevacor), pravastatin (e.g., Pravachol), niacin; antihypertensives, such as propranol, ACE inhibitos (e.g., lisinopril (e.g., Prinivil, Zestril), calcium channel blockers, i.e., nifedipine (e.g., Adalat, Procardia), diuretics; as well as medications used to treat other conditions, such as hematology applications, other neurodegenerative disorders, such as multiple sclerosis, Niemann-Pick Type C, stroke, ocular conditions, such as glaucoma and macular degeneration, and cancer (e.g., Gleevec).

The effective amount of any modified neuroactive steroid, the use of which is in the scope of present disclosure will vary somewhat from agent to agent, and patient to patient, and will depend upon factors such as the age and condition of the patient and the route of delivery. Such dosages can be determined in accordance with routine pharmacological procedures known to those skilled in the art. As a general proposition, a dosage from about 0.1 to about 50 mg/kg will have therapeutic efficacy, with all weights being calculated based upon the weight of the patient. Toxicity concerns at the higher level may restrict intravenous dosages to a lower level such as up to about 10 mg/kg, with all weights being calculated based upon the weight of the active base. A dosage from about 10 mg/kg to about 50 mg/kg may be employed for oral administration. Typically, a dosage from about 0.5 mg/kg to 5 mg/kg may be employed for intramuscular injection. The frequency of administration is usually one, two, or three times per day or as necessary to control the condition. The duration of treatment depends on the type of condition being treated and may be for as long as the life of the patient.

The present disclosure is explained in greater detail in the following non-limiting examples.

### EXAMPLES

### Example

### Preparation of Neuroactive Steroids Illustrated in Table 1

Ester derivatives at the C3 positions may be obtained by treating the neuroactive steroid, such as pregnenolone, with an acid chloride derivative or a carboxylic acid in the presence of a coupling reagent such as N,N′-Dicyclohexylcarbodiimide (DCC) to prepare the modified neuroactive steroid.

A. Esters at C3

Esters at C3

Specific examples

B. Preparation of enol esters

Preparation Of Enol Esters

C. Preparation of diesters

Preparation of Diesters

One skilled in the art will readily appreciate that the present invention is well adapted to carry out the objects and obtain the ends and advantages mentioned, as well as those inherent therein. The present examples along with the methods, procedures, treatments, molecules, and specific compounds described herein are presently representative of preferred embodiments, are exemplary, and are not intended as limitations on the scope of the invention. Changes therein and other uses will occur to those skilled in the art which are encompassed within the spirit of the invention as defined by the scope of the claims.

Any patents or publications mentioned in this specification are indicative of the levels of those skilled in the art to which the invention pertains. These patents and publications are herein incorporated by reference to the same extent as if each individual publication was specifically and individually indicated to be incorporated by reference.

1-38. (canceled)

39. A modified neuroactive steroid having the general formula R-Lₙ-NS, wherein R is selected from the group consisting of H, a neuroactive steroid, a therapeutic agent, a modifying moiety, and combinations thereof; L is a linker; n is an integer equal to or greater than 1; and NS is a neuroactive steroid, wherein said linker is selected from the group consisting of:
wherein w is 0-20; m is 2-20; t is 1-20; U is —O—(C═O)—R′, —NH—(C═O)—R′, —NR*—(C═O)—R′, —OH, or —NH₂; V is —H or —CH₃; D is —H, —CH₃, —CO₂H, —CO₂R′, —CHCO₂H, or —CH₂CO₂R′; R* is H or a lower alkyl; and R′ is independently selected from a neuroactive steroid, R, an alkyl, and a linker.

40. The modified neuroactive steroid of claim 39, wherein n is an integer from 1 to 50.

41. The modified neuroactive steroid of claim 39, wherein NS is selected from the group consisting of pregnenolone, allopregnanolone, epiallopregnanolone, epipregnanolone, progesterone, 3α-hydroxyprogesterone, 3β-hydroxyprogesterone, 5α-dihydroprogesterone, 5β-dihydroprogesterone, androsterone, dehydroepiandrosterone, allotetrahydrodeoxycorticosterone, 3α,5α-cortisol, 3α,5β-cortisol, 3α,5α-11-deoxycortisol, 3α,5β-11-deoxycortisol, 5α-dihydrocortisol, 5β-dihydrocortisol, and pharmaceutically acceptable salts thereof, derivatives thereof, or combinations thereof.

42. The modified neuroactive steroid of claim 39, having a structure selected from the group consisting of:
wherein R₂ is ═O or —OH; R₃ is —H or —OH; and R₄ is —H or —OH, and ----- is an optional C═C bond.

43. The modified neuroactive steroid of claim 39, wherein R is a neuroactive steroid.

44. The modified neuroactive steroid of claim 43 having a structure selected from the group consisting of:

45. The modified neuroactive steroid of claim 43, wherein R is selected from the group consisting of pregnenolone, allopregnanolone, epiallopregnanolone, epipregnanolone, progesterone, 3α-hydroxyprogesterone, 3β-hydroxyprogesterone, 5α-dihydroprogesterone, 5β-dihydroprogesterone, androsterone, dehydroepiandrosterone, allotetrahydrodeoxycorticosterone, 3α,5α-cortisol, 3α,5β-cortisol, 3α,5α-1′-deoxycortisol, 3α,5β-1′-deoxycortisol, 5α-dihydrocortisol, 5β-dihydrocortisol, and pharmaceutically acceptable salts thereof, derivatives thereof, or combinations thereof.

46-51. (canceled)

52. The modified neuroactive steroid of claim 39 having a structure selected from the group consisting of:

53-55. (canceled)

56. A modified neuroactive steroid having the general formula R-Lₙ-NS, wherein R is selected from the group consisting of H, a neuroactive steroid, a therapeutic agent, a modifying moiety, and combinations thereof; L is a sulfur- or phosphorus-containing linker; n is an integer equal to or greater than 1; and NS is a neuroactive steroid, wherein said linker is selected from the group consisting of:
wherein w is 0-20; m is 2-20; t is 1-20; u is 1 or 2; s is 1 or 2; y is 0 or 1; z is 0 or 1; U is —O—X(O)ᵤ—(O_{y}R′)ₛ, —NH—X(O)ᵤ—(O_{y}R′)ₛ, —NR*—X(O)ᵤ—(O_{y}R′)ₛ, —OH, or —NH₂; V is —H or —CH₃; D is —H, —CH₃, —CO₂H, —CO₂R′, —CHCO₂H, or —CH₂CO₂R′; R* is —H or a lower alkyl; R′ is independently selected from a neuroactive steroid, R, an alkyl, and a linker; X is C, S, or P, with the proviso that at least one X in the linker is S or P; R₅, when present, is independently selected from ═O and —OR₇; R₆ is independently selected from —H and —OR₇; and R₇ is —H or R′.

57. The modified neuroactive steroid of claim 56, wherein n is an integer from 1 to 50.

58. The modified neuroactive steroid of claim 56, wherein NS is selected from the group consisting of pregnenolone, allopregnanolone, epiallopregnanolone, epipregnanolone, progesterone, 3α-hydroxyprogesterone, 3β-hydroxyprogesterone, 5α-dihydroprogesterone, 5β-dihydroprogesterone, androsterone, dehydroepiandrosterone, allotetrahydrodeoxycorticosterone, 3α,5α-cortisol, 3α,5β-cortisol, 3α,5α-11-deoxycortisol, 3α,5α-11-deoxycortisol, 5α-dihydrocortisol, 5β-dihydrocortisol, and pharmaceutically acceptable salts thereof, derivatives thereof, or combinations thereof.

59. The modified neuroactive steroid of claim 56, wherein R is a neuroactive steroid selected from the group consisting of pregnenolone, allopregnanolone, epiallopregnanolone, epipregnanolone, progesterone, 3α-hydroxyprogesterone, 3β-hydroxyprogesterone, 5α-dihydroprogesterone, 5β-dihydroprogesterone, androsterone, dehydroepiandrosterone, allotetrahydrodeoxycorticosterone, 3α,5α-cortisol, 3α,5β-cortisol, 3α,5α-11-deoxycortisol, 3α,5β-11-deoxycortisol, 5α-dihydrocortisol, 5β-dihydrocortisol, and pharmaceutically acceptable salts thereof, derivatives thereof, or combinations thereof.

60. (canceled)

61. (canceled)

62. The modified neuroactive steroid of claim 56, having a structure selected from the group consisting of:
wherein R₂ is ═O or —OH; R₃ is —H or —OH; and R₄ is —H or —OH, and ----- is an optional C═C bond.

63. A modified neuroactive steroid having the general formula R-Lₙ-NS, wherein R is selected from the group consisting of a neuroactive steroid, a therapeutic agent, a modifying moiety, and combinations thereof; L is a sulfur- or phosphorus-containing linker; n is an integer equal to or greater than 1; and NS is a neuroactive steroid.

64. The modified neuroactive steroid of claim 63, wherein n is an integer from 1 to 50.

65. The modified neuroactive steroid of claim 63, wherein NS is selected from the group consisting of pregnenolone, allopregnanolone, epiallopregnanolone, epipregnanolone, progesterone, 3α-hydroxyprogesterone, 3β-hydroxyprogesterone, 5α-dihydroprogesterone, 5β-dihydroprogesterone, androsterone, dehydroepiandrosterone, allotetrahydrodeoxycorticosterone, 3α,5α-cortisol, 3α,5β-cortisol, 3α,5α-1′-deoxycortisol, 3α,5β-11-deoxycortisol, 5α-dihydrocortisol, 5β-dihydrocortisol, and pharmaceutically acceptable salts thereof, derivatives thereof, or combinations thereof.

66. The modified neuroactive steroid of claim 63, wherein R is a neuroactive steroid selected from the group consisting of pregnenolone, allopregnanolone, epiallopregnanolone, epipregnanolone, progesterone, 3α-hydroxyprogesterone, 3β-hydroxyprogesterone, 5α-dihydroprogesterone, 5β-dihydroprogesterone, androsterone, dehydroepiandrosterone, allotetrahydrodeoxycorticosterone, 3α,5α-cortisol, 3α,5β-cortisol, 3α,5α-11-deoxycortisol, 3α,5β-11-deoxycortisol, 5α-dihydrocortisol, 5β-dihydrocortisol, and pharmaceutically acceptable salts thereof, derivatives thereof, or combinations thereof.

67. (canceled)

68. (canceled)

69. The modified neuroactive steroid of claim 63, having a structure selected from the group consisting of:
wherein R₂ is ═O or —OH; R₃ is —H or —OH; and R₄ is —H or —OH, and is an optional C═C bond.

70. The modified neuroactive steroid of claim 63 comprising a structure selected from the group consisting of:

71. The modified neuroactive steroid of claim 63 having a structure selected from the group consisting of:

72-80. (canceled)
